(19) United States
(12) Patent Application Publication
(10) Pub. No.: US 2018/0162915 A1
(43) Pub. Date: Jun. 14, 2018

(54) METHODS AND COMPOSITIONS FOR MODIFYING PLANT ARCHITECTURE AND DEVELOPMENT

(71) Applicant: PIONEER HI-BRED INTERNATIONAL, INC., JOHNSTON, IA (US)

(72) Inventors: XIAOMU NIU, JOHNSTON, IA (US); JOAN MARIE PHILLIPS, PROLE, IA (US); MARY TRIMNELL, WEST DES MOINES, IA (US)

(73) Assignee: PIONEER HI-BRED INTERNATIONAL, INC., JOHNSTON, IA (US)

(21) Appl. No.: 15/577,387

(22) PCT Filed: May 27, 2016
(86) PCT No.: PCT/US16/34534

§ 371 Date: Nov. 28, 2017

Related U.S. Application Data
(60) Provisional application No. 62/167,362, filed on May 28, 2015.

(51) Int. Cl. C07K 14/415; C12N 15/82

(52) U.S. Cl. CPC C07K 14/415; C12N 15/827; C12N 15/8266; C12N 15/8261

(57) ABSTRACT

Methods and compositions useful for modifying plant architecture and development are provided herein. Plants with altered levels of MATE-efflux polypeptides that exhibit altered agronomic characteristics are provided. Nucleotide and polypeptide sequences of members of MATE-efflux family, along with recombinant DNA constructs useful for conferring altered agronomic characteristics upon plants comprising these sequences, are also provided.

### REFERENCE TO SEQUENCE LISTING SUBMITTED ELECTRONICALLY

The official copy of the sequence listing is submitted electronically via EFS-Web as an ASCII formatted sequence listing with a file named “5190USPSP_SequenceListing” created on May 27, 2015 and having a size of 86 kilobytes and is filed concurrently with the specification. The sequence listing contained in this ASCII formatted document is part of the specification and is herein incorporated by reference in its entirety.

### FIELD

The field relates to plant breeding and genetics and, in particular, relates to recombinant DNA constructs useful for modulation of plant architecture and development.

### BACKGROUND

Plant architecture and development are key factors that affect plant survival and productivity. Plant architecture, which is the three-dimensional organization of the plant body, is of major agronomic importance, strongly influencing the suitability of a plant for cultivation, its yield and the efficiency with which it can be harvested. Plant architecture includes many agronomically important traits such as branching pattern, root and shoot diameter, size, number, position and shape of leaves and flower organs.

Growth, development and the duration of life cycle of an agronomic ally important plant also greatly influences productivity, and tolerance to various environmental conditions. For example, days to shed, days to silk, and flowering time are important for optimizing grain yield in corn. Flowering time also determines maturity, which is an important agronomic trait. Manipulating other traits such as grain moisture content can also have effects on yield. Low (or reduced) grain moisture decreases the economic impact of artificial drying and allows earlier harvesting, which permits the grower to obtain a higher price for the crop at an earlier date and reduced exposure of the crop to adverse weather and field conditions that hinder the harvest operation. Plants can also respond to stress and to other environmental conditions by adapting metabolic activity and growth rate (Reinhardt and Kuhlemeier, 2002 EMBO reports; 3 (9):846-851; Li et al (2014) Plos Genetics 10(1) e1003954; Doleferus R. Plant Science 229 (2014) 247-261; Sweeney et al (1994) Crop Sci 34: 391-396).

Identifying genes that contribute to regulating agronomic traits associated with plant growth and architecture can contribute to increasing crop productivity in various environments.

### SUMMARY

The present disclosure includes:

One embodiment of the current disclosure is a plant comprising in its genome a recombinant DNA construct comprising a polynucleotide operably linked to at least one heterologous regulatory element, wherein said polynucleotide encodes a MATE-efflux polypeptide, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising said recombinant DNA construct. In one embodiment, the polynucleotide encodes a MATE-efflux polypeptide having an amino acid sequence of at least 80% sequence identity, when compared to SEQ ID NO:2, 4, 6, 8, 10, 12, 13, 14, 15, 16, 17, 18 or 20. In one embodiment, the polynucleotide encodes a MATE-efflux polypeptide comprising the amino acid sequence of SEQ ID NO:2, 4, 6, 8, 10, 12, 13, 14, 15, 16, 17, 18 or 20. In one embodiment, the plant overexpresses said polypeptide. In one embodiment, the maturity of plant is reduced.

One embodiment is a plant comprising in its genome a recombinant DNA construct comprising a polynucleotide operably linked to at least one heterologous regulatory element, wherein said polynucleotide encodes a MATE-efflux polypeptide, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising said recombinant DNA construct, wherein the polynucleotide comprises a nucleotide sequence that has at least 80% sequence identity, when compared to SEQ ID NO:1, 3, 5, 7, 9 or 19, and wherein the polynucleotide sequence can be modified by Cas9 nuclease/guide-RNA mediated genome editing approach.

Another embodiment is a plant comprising in its genome an endogenous polynucleotide operably linked to at least one heterologous regulatory element, wherein said endogenous polynucleotide encodes a MATE-efflux polypeptide having an amino acid sequence that has at least 80% sequence identity, when compared to SEQ ID NO:2, 4, 6, 8, 10, 12, 13, 14, 15, 16, 17, 18 or 20, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising the heterologous regulatory element operably linked to the endogenous polynucleotide. In one embodiment, the at least one heterologous regulatory element is at least one regulatory element endogenous to the plant.

Another embodiment is a method of conferring upon a plant at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising increasing the expression of a MATE-efflux protein in the plant.

One embodiment is a method of conferring upon a plant at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising the steps of (a) introducing into a regenerable plant cell a recombinant DNA construct comprising a polynucleotide operably linked to at least one heterologous regulatory sequence, wherein the polynucleotide encodes a MATE-efflux polypeptide having an amino acid sequence that has at least 80% sequence identity, when compared to SEQ ID NO:2, 4, 6, 8, 10, 12, 13, 14, 15, 16, 17, 18 or 20; (b) regenerating a transgenic plant from the regenerable plant cell of (a), wherein the transgenic plant comprises in its genome the recombinant DNA construct; and (c) obtaining a progeny plant derived from the transgenic plant of (b), wherein said progeny plant comprises in its genome the recombinant DNA construct and exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the recombinant DNA construct.

One embodiment is a method of selecting a plant that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising: (a) obtaining a transgenic plant, wherein the transgenic plant comprises in its genome a recombinant DNA construct comprising a polynucleotide operably linked to at least one heterologous regulatory element, wherein said polynucleotide encodes a MATE-efflux polypeptide having an amino acid sequence that has at least 80% sequence identity, when compared to SEQ ID NO:2, 4, 6, 8, 10, 12, 13, 14, 15, 16, 17, 18 or 20; (b) growing the transgenic plant of part (a) under conditions wherein the polynucleotide is expressed; and (c) selecting the transgenic plant of part (b) with at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the recombinant DNA construct.

In one embodiment, the plant disclosed herein is a monocot plant. In one embodiment, the monocot plant is a maize plant.

One embodiment of the current disclosure is a method of increasing yield of a crop plant, the method comprising increasing expression of a MATE-efflux protein in the crop plant. In one embodiment, the crop plant is planted at a density higher than a control crop plant.

One embodiment is method of selecting a plant that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising the steps of: (a) introducing a mutation into an endogenous MATE-efflux gene of a plant, to create a mutant plant comprising a MATE-efflux mutant gene; and (b) selecting the mutant plant of step (a) that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the MATE-efflux mutant gene. In one embodiment, step (a) is done using at least one method selected from the group consisting of: Targeting Induced Local Lesions IN Genomics (TILLING), transposon tagging, and Cas9 nuclease/guide-RNA mediated genome editing technology. In one embodiment, the mutation is in a non-coding region of the MATE-efflux gene.

In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The Cas9/guide RNA-based system allows targeted cleavage of genomic DNA guided by a customizable small noncoding RNA in plants (see e.g., WO 2015026883A1).

Another embodiment is a recombinant DNA construct comprising a polynucleotide operably linked to at least one heterologous regulatory element, wherein said polynucleotide encodes a MATE-efflux polypeptide, and wherein said recombinant DNA construct confers upon a plant comprising said recombinant DNA construct at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising said recombinant DNA construct.

One embodiment is a recombinant DNA construct comprising a polynucleotide operably linked to at least one heterologous regulatory element, wherein said polynucleotide encodes a MATE-efflux polypeptide having an amino acid sequence that has at least 80% sequence identity, when compared to SEQ ID NO:2, 4, 6, 8, 10, 12, 13, 14, 15, 16, 17, 18 or 20, and wherein said recombinant DNA construct confers upon a plant comprising said recombinant DNA construct at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising said recombinant DNA construct.

In one embodiment, the current disclosure encompasses seed of any of the plants disclosed herein. In one embodiment, the seed comprises in its genome a recombinant DNA construct comprising a polynucleotide operably linked to at least one heterologous regulatory element, wherein said polynucleotide encodes a MATE-efflux polypeptide having an amino acid sequence of at least 80% sequence identity, when compared to SEQ ID NO:2, 4, 6, 8, 10, 12, 13, 14, 15, 16, 17, 18 or 20, and wherein a plant produced from said seed exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising said recombinant DNA construct.

In another embodiment, the present disclosure includes any of the methods of the present disclosure wherein the plant is selected from the group consisting of: Arabidopsis, maize, soybean, sunflower, sorghum, canola, wheat, alfalfa, cotton, rice, barley, millet, sugar cane and switchgrass.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1A-FIG. 1E show the alignment of the MATE-efflux polypeptides given in SEQ ID NOS:2, 4, 6, 8, 10-18 and 20. Residues that are identical to the residue of SEQ ID NO:2) at a given position are enclosed in a box. A consensus sequence (SEQ ID NO:21) is presented where a residue is shown if identical in all sequences, otherwise, a period is shown.

FIG. 2 shows the percent sequence identity and the divergence values for each pair of amino acids sequences of MATE efflux polypeptides displayed in FIG. 1A-1E.

FIG. 3 shows the effect on plant development in a MATE9 mutant plant and in a MATE9 overexpressing maize plant compared to wild-type and a MATE9 non-expressing plant. Leaf count, time to shed (GDUSHD), time to silk (GDUSLK) and leaf appearance rate are compared. All data was collected from field conditions except for: the leaf count for the mutant and the wild-type plant, that was collected from plants grown in a 16-hr day growth chamber, indicated by a single asterisk; and the leaf appearance rate for the mutant and the wild-type plant, that was collected from plants grown in a greenhouse indicated by a double asterisk. CRM stands for corn relative maturity.

FIG. 4 shows the time to shed (GDUSHD), time to silk (GDUSLK), grain moisture content and yield analysis of maize lines transformed with pUbi_ZmMATE9 encoding the maize MATE9 polypeptide (SEQ ID NO:2). GDUSHD, GDUSLK and grain moisture content are shown as differences from the bulk null values. Yield is shown as a percent difference from the bulk null.

FIG. 5A shows the analysis of moisture content in plants comprising the pUbi_AtMATE_EP1 construct and overexpressing the At-MATE_EP1 polypeptide (SEQ ID NO:20). Different locations with different stress levels are shown as LS (low stress), MS (medium stress) and SS (severe stress).

FIG. 5B shows the yield analysis in plants comprising the pUbi_AtMATE_EP1 construct and overexpressing the At-MATE_EP1 polypeptide (SEQ ID NO:20). Different locations with different stress levels are shown as LS (low stress), MS (medium stress) and SS (severe stress). Yield is shown as percent difference from the bulk null values.

FIG. 6A-C show the analysis of agronomic traits such as ear height (EARHT), time to shed (GDUSHD), time to silk (GDUSLK), grain moisture content, plant height (PLTHT) in plants comprising the pUbi_ZmMATE_EP1 construct and overexpressing the ZmMATE_EP1 polypeptide (SEQ ID NO:6). FIG. 6A shows ear height (EARHT), time to shed (GDUSHD), time to silk (GDUSLK) at locations with different drought stress conditions, with flowering stress, and optimal or no-stress conditions. The values are the difference from the bulk null values. The statistically significant values are shown in bold. FIG. 6B shows grain moisture content at four different locations, one each with flowering stress, grain filling stress and two locations with optimal or no-stress conditions. The figure also shows plant height (PLTHT) at one flowering stress location. The bulk null values and the values for different transgenic events are shown. The statistically significant values are shown in bold. FIG. 6C shows yield analysis at four different locations, one each with flowering stress, grain filling stress and two locations with optimal or no-stress conditions. The percent difference values from bulk null are shown for different transgenic events. The statistically significant values are shown in bold.

Table 1 presents SEQ ID NOs for the MATE-efflux nucleotide and protein sequences from Zea mays, Sorghum, Setaria italica, Hordeum vulgare, Brachypodium distachyon, Oryza sativa and Arabidopsis thaliana.

TABLE 1
MATE-efflux proteins

| | | SEQ ID | SEQ ID NO: |
|---|---|---|---|
| | Clone Designation/ | NO: | (Amino |
| Plant | NCBI GI No. | (Nucleotide) | Acid) |
| Corn | ZmMATE9 | 1 | 2 |
| Corn | ZmMATE7 | 3 | 4 |
| Corn | ZmMATE_EP1 | 5 | 6 |
| Corn | ZmMATE7_like | 7 | 8 |
| Corn | NM_001174889 | 9 | 10 |
| Hordeum vulgare | GI No. 326515342 | — | 11 |
| Brachypodium | GI No. 357154343 | — | 12 |
| distachyon | | | |
| Oryza sativa | GI No. 125546368 | — | 13 |
| Setaria italica | GI No. 514812645 | — | 14 |
| Sorghum bicolor | GI No. 242037467 | — | 15 |
| Hordeum vulgare | GI No. 545693653 | — | 16 |
| Oryza sativa | GI No. 297609831 | — | 17 |
| Sorghum bicolor | GI No. 242049900 | — | 18 |
| Arabidopsis thaliana | At_MATE_EP | 19 | 20 |

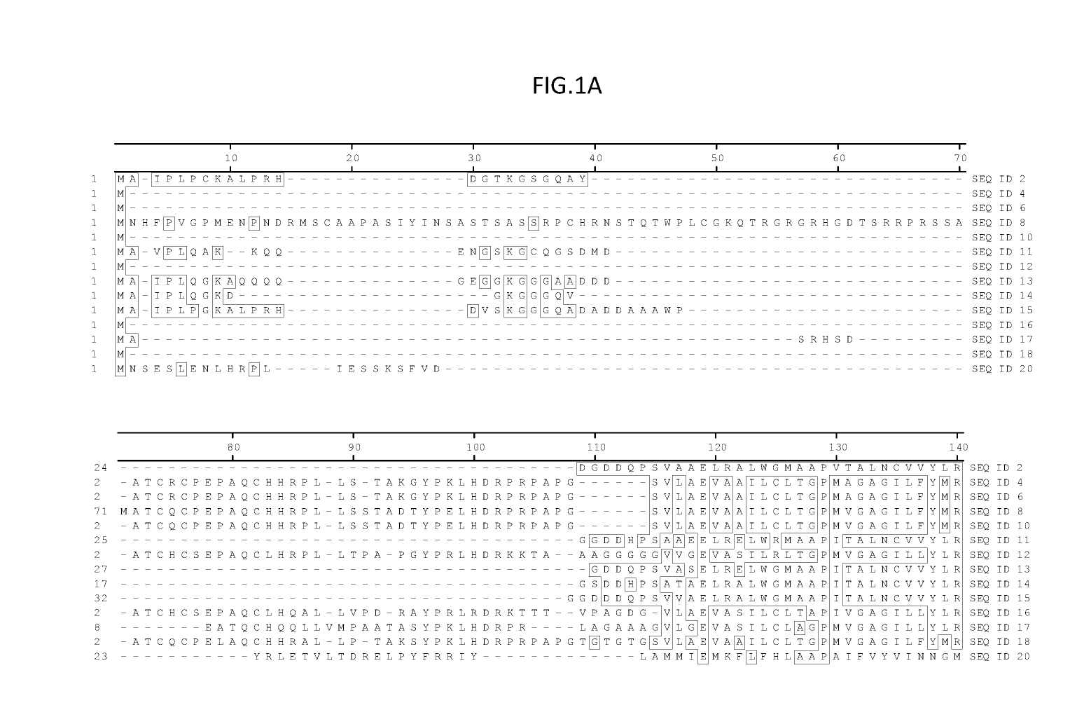

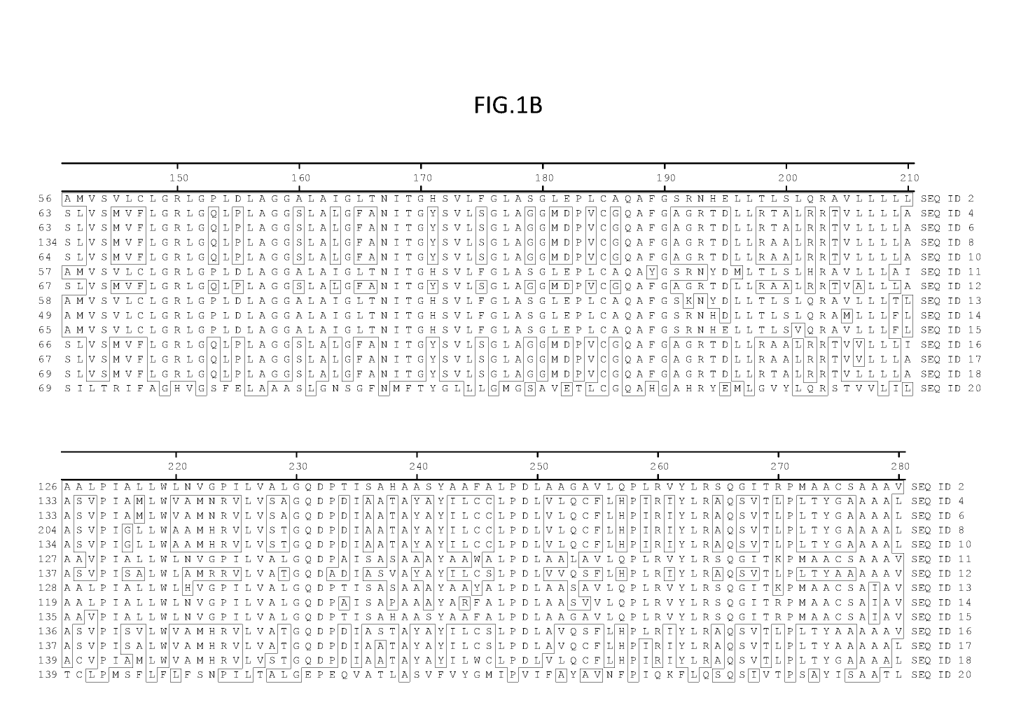

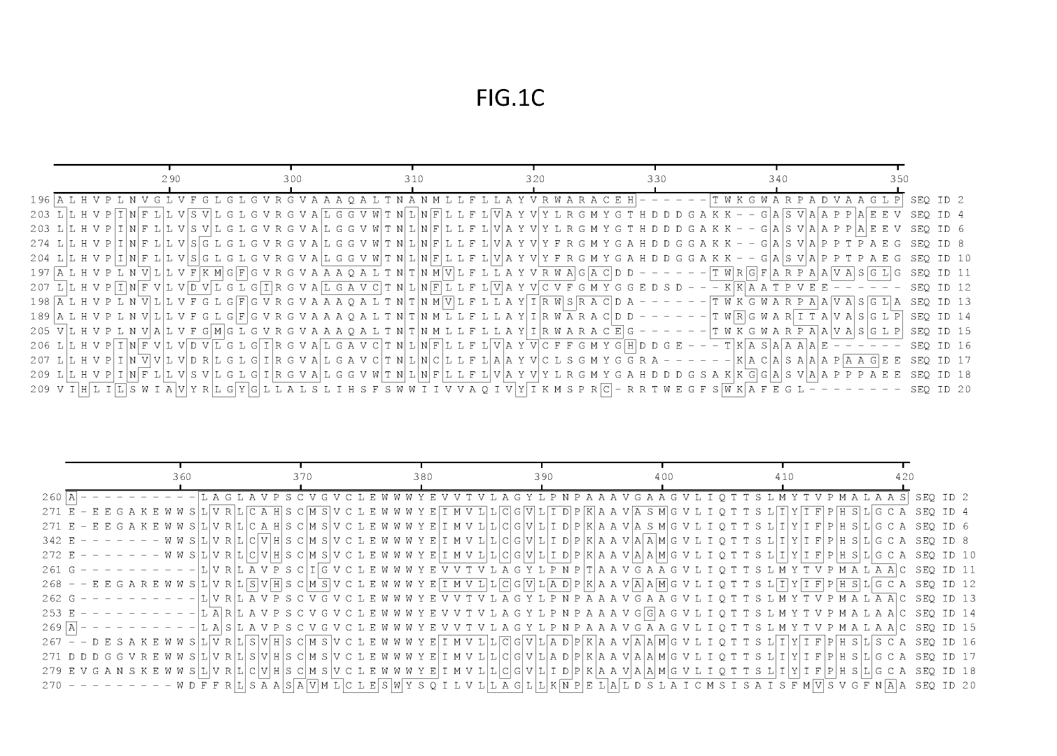

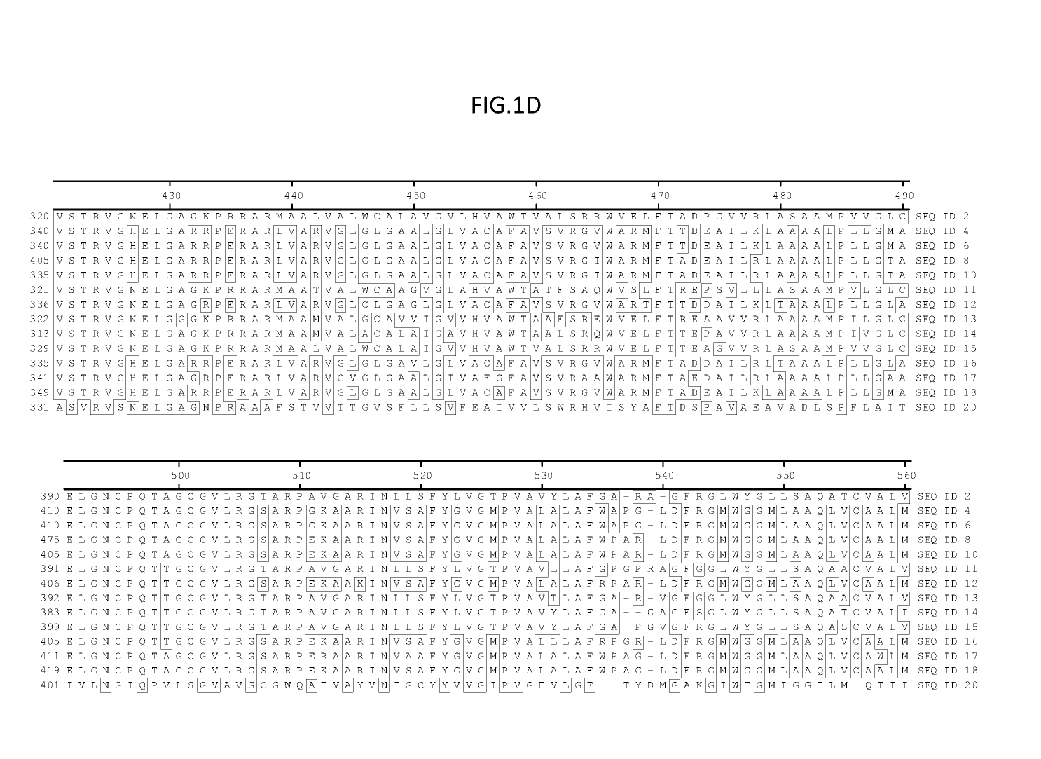

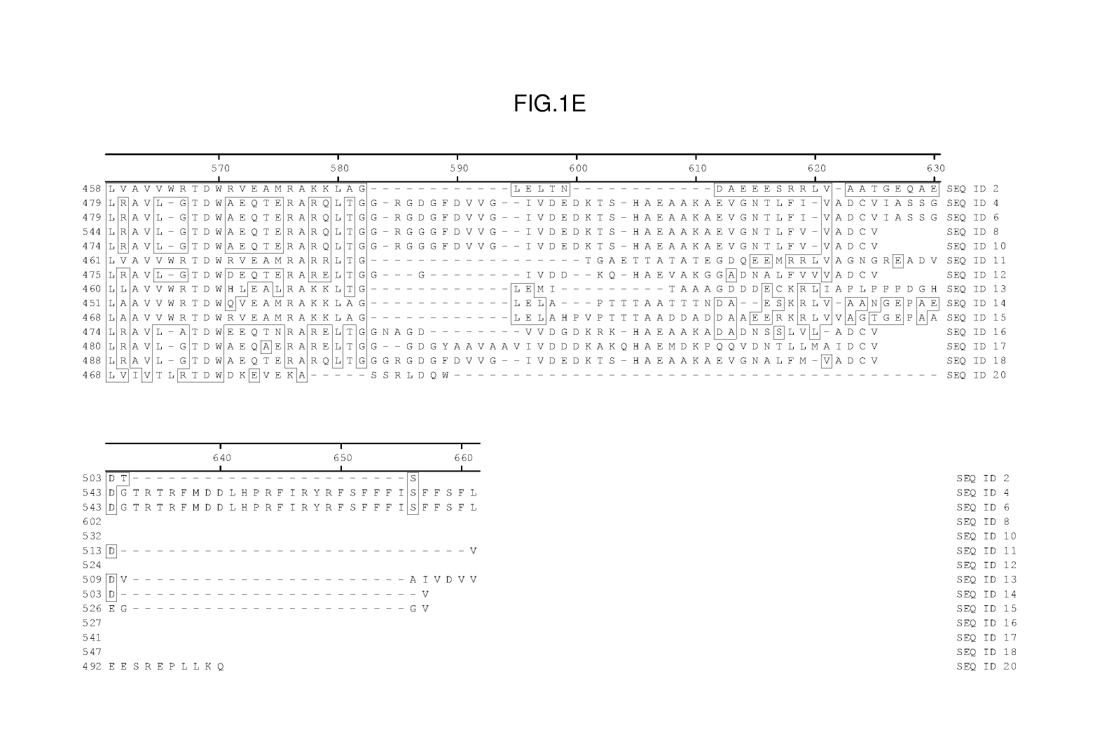

SEQ ID NO:21 is the consensus sequence obtained by aligning the MATE-efflux polypeptides as shown in FIG. 1A-1E.

The sequence descriptions and Sequence Listing attached hereto comply with the rules governing nucleotide and/or amino acid sequence disclosures in patent applications as set forth in 37 C.F.R. § 1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. § 1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The terms “MATE-EP”, “MATE-efflux protein”, “MATE protein” and “Multidrug and Toxic compound Extrusion proteins” are used interchangeably herein.

The term “MATE” stands for “Multidrug and Toxic compound Extrusion”; these two terms are used interchangeably herein.

Toxins and secondary metabolites are removed from the plant cytoplasm and stored in the vacuole or the cell wall. The compounds that need to be sequestered can be produced endogenously, such as flavonoids, or could be xenobiotics. MATE proteins are a recently identified family of multidrug transporters and are secondary transport proteins and are characterized by 400-700 amino acids and twelve predicted transmembrane domains. Members of this family have been found in all kingdoms of living organisms. There are 58 family members known in Arabidopsis, based on sequence homology (Omote et al. (2006) Trends Pharmaceutical Sci. 27(11): 587-593). Multidrug and toxic compound extrusion transporters represent a large family in plants, but their functions are poorly understood. The Plant MATEs characterized so far have been found to be involved in the detoxification of endogenous secondary metabolites and xenobiotics (Brown et al. (1999) Molecular microbiology 31(1):393-395, Eckardt N A (2001) Plant Cell 13: 1477-1480). Some MATE proteins are involved in the transport of citrate, which is required for iron (Fe) translocation or aluminum detoxification (Yokosho et al, Plant Physiology, January 2009 (149), pp. 297-305, PCT Publication No. WO2014151749, US Patent Publication No. US20140298542).

ALF5, EDS5 and TRANSPARENT TESTA 12 (Tt12) encode Arabidopsis MATE proteins (Omote et al (2006) Trends Pharmaceutical Sci. 27(11): 587-593; Nawrath et al. (2002) Plant Cell 14: (275-286); Diener et al. (2001) Plant cell 13:1625-1637). Li et al have shown that ADP1, a putative MATE polypeptide from Arabidopsis plays an essential role in maintaining normal architecture in Arabidopsis (Li et al PLOS Genetics January 2014 (10:1: e1003954).

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” generally refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic stress tolerance, greenness, yield, growth rate, biomass, fresh weight at maturation, dry weight at maturation, fruit yield, seed yield, grain moisture content, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height or stature, leaf number, time to flowering, days to shed, days to silk, time for grain filling and time for grain dry down, plant maturity, leaf appearance rate, ear height, ear length, salt tolerance, early seedling vigor and seedling emergence under low temperature stress.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS).

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

As used herein, “grain moisture” is a property of grain that is measured in order to determine the optimal time to harvest. It can be measured using any method, example of which includes, but is not limited to, using a meter that measure the electrical properties of the grain (PCT Publication No. WO2013/126689).

The term “planting density” or “plant density” as used herein is defined as the number of plants per unit area, The area may be measured in acres or hectares.

High plant densities and narrow rows lead to increased leaf area index, allowing interception of more of the light energy reaching the earth's surface.

“Leaf area index” or “LAI” is defined herein as leaf area per unit land area.

“Lodging” is defined as the falling over of plants.

“Leaf appearance rate” is the inverse of the time duration that's separates the appearance of two successive leaves, and can be measured by days or heat units it takes to add a leaf (Bonhomme R. Eur J Agronomy 13 (2000) 1-10).

Plant senescence is a developmental process which in annual crop plants overlaps with the reproductive phase, and is the final stage of plant development, wherein mineral nutrients are mobilized and translocated to the maturing storage organ from vegetative plant parts that eventually die off (Gregersen and Culetic Plant Mol Biol (2013) 82:603-622).

“Stay-green” or “staygreen” is a term used to describe a plant phenotype, e.g., whereby leaf senescence (most easily distinguished by yellowing of leaf associated with chlorophyll degradation) is delayed compared to a standard reference or a control.

Maturity of a plant generally refers to the duration between the planting of seeds to harvesting grains. During this process, plants go through three major stages time to flowering, grain filling and dry down. Time to flowering includes seed planting, emergence through anthesis all of which are vegetative growth. During this stage, plants accumulate biomass and establish canopy growth. Grain filling is the second main stage, when plants are actively depositing photosynthates into growing grains from post-anthesis to physiological maturity. Physiological maturity of a crop plant describes that stage when sexually induced reproductive growth has ceased (Burns H. A. (2009) Agronomy Journal 101 (1):60-66; PCT Publication No. WO2014160304).

Grain corn can be harvested only after it reaches a certain level of grain moisture, which can be around 20-40%.

Since maturity includes all 3 stages, shortening anyone or more stages would result in an overall reduction in maturity. One or more of the following technical approaches achieve shortened maturity: reducing days to shed and silk (flowering), accelerating grain filling or decreasing duration for dry down.

Grain moisture is an important trait for maize production. If the grain is too moist when the grower wants to harvest, then the grower may have to leave the crop in the field for a longer period of time, thereby exposing the crop to adverse weather and field conditions that could affect yield. Furthermore, once the grain is harvested, artificial drying may be needed to achieve a desired grain moisture level, requiring access to drying equipment, transportation to move the grain to the dryers, and power to run the dryers (Sweeney et al. (1994) Crop Science 34:391-396; Brown and Bootsma (2002) Can. J. Plant Sci. 82: 549-550

Plants that mature earlier, tolerate higher population densities, with low grain moisture contents, can be useful for short season areas, and can also be useful for increasing productivity (Begna et al. (1997) J. Agronomy & Crop Science 179, 9-17). Planting at higher densities can be used for increasing crop yield. Total leaf area index can be increased by increasing planting density. It has been shown that density tolerant maize hybrids are characterized by traits such as rapid completion of silk extrusion, growth of first ear and first appearance of ear silk (Begna et al (1997) J. Agronomy & Crop Science 179, 9-17)

The growth and emergence of maize silks has a considerable importance in the determination of yield under drought (Fuad-Hassan et al. 2008 Plant Cell Environ. 31:1349-1360). When soil water deficit occurs before flowering, silk emergence out of the husks is delayed while anthesis is largely unaffected, resulting in an increased anthesis-silking interval (ASI) (Edmeades et al. 2000 Physiology and Modeling Kernel set in Maize (eds M. E. Westgate & K. Boote; CSSA (Crop Science Society of America) Special Publication No. 29. Madison, Wis.: CSSA, 43-73). Selection for reduced ASI has been used successfully to increase drought tolerance of maize (Edmeades et al. 1993 Crop Science 33: 1029-1035; Bolanos & Edmeades 1996 Field Crops Research 48:65-80; Bruce et al. 2002 J. Exp. Botany 53:13-25).

Heat units (HU) are used to describe thermal time, and explain temperature impact on rate of corn development, and these HUs provide growers an indexing system for selection of corn hybrids in a given location. Several formulas exist for the calculation of heat units. Among them, GDD or GDU (Growing Degree Day or Growing Degree Unit) and CHU (Crop Heat Units) are most commonly used.

The terms “growing degree days” (GDD), “growing degree units” (GDU) are used interchangeably herein. Growing degree days are often accumulated over a specified number of days. The GDD is usually accumulated from the day of planting until a specific developmental stage such as shedding/silking and/or maturity. The GDD calculation for corn is generally well known (Burns H. A. (2009) Agronomy Journal 101 (1) “Wiebold B., “Growing Degree Days and Corn Maturity” MU Plant Sciences Extension Web Site; Nielsen et al Agron. J. (2002) 94:549-558).

GTI (General Thermal Index) has recently been developed that attempts to improve accuracy in predicting developmental stages.

The methods to calculate GDD and HUs are well known in the art. The method to calculate GDD is to average daily temperature (degrees F.) then minus 50, proposed by the National Oceanic and Atmospheric Administration and labeled as the “Modified Growing Degree Day”.

GDU=(Tmax+Tmin)/2−Tbase

Where T max is maximum daily temperature, T min is minimum daily temperature, and Tbase is a base temperature (mostly set at 50 F).

The method to calculate CHU is somewhat more complex, allocating different responses of development to temperature (degrees C.) between the day and the night.

CHUday=3.33*(Tmax−10)−0.084*(Tmax−10)2

CHUnight=1.8*(Tmin−4.4)

CHU=[CHUday+CHUnight]/2

GTIs are calculated based on different responses of corn from planting to silking and from silking to maturity. The period between planting and silking is defined as vegetative growth, whereas time from silking to maturity is the grain filling stage.

FT(veg)=0.0432T2−0.000894T3

FT(fill)=5.358+0.011178T2

GTI=FT(veg)+FT(fill)

Where T is mean daily temperature (degrees C.), FT(veg) is for the period from planting to silking, FT(fill) is for the period from silking to maturity.

Relative Maturity Conversion Guidelines

Guidelines for converting various relative-maturity rating systems have been reported by Dwyer, et a/., (Agron. J. (1999) 91:946-949). Conversions for CHU, GDD and the Corn Relative Maturity rating system (CRM), also referred to as the Minnesota Relative Maturity Rating, are generally available. The CRM rating system is widely used in the US to characterize hybrid relative maturity. The CRM rating is not based on temperature, but on the duration in days from planting to maturity (in an average year) relative to a set of standard hybrids. The approximate conversion from one rating system to another can be estimated from a linear regression equation.

Maturity may also generally refer to a physiological state, where maximum weight per kernel has been achieved for the planted corn. This is often referred to as physiological maturity and is generally associated with the formation of an abscission layer or “black layer” at the base of the kernel. One of the most commonly used methods for designating hybrid maturity ratings (days to maturity) is based on comparisons among hybrids close to the time of harvest.

Kernel dry weight does not generally increase beyond physiological maturity. Kernel drying that occurs following black layer is mostly due to evaporative moisture loss. Drydown rates are generally the greatest during the earlier, warmer part of the harvest season and decline as the weather gets colder.

“Harvest maturity” is a function of grain moisture percentage. Since grain drying adds to the cost of corn production and, therefore, grain moisture at harvest is the dominant feature is assessing hybrid maturity.

“Harvest index” is the ratio of the grain dry weight to total aboveground dry weight (biomass) of a crop at maturity, is an indicator of dry matter partitioning efficiency.

Increased planting density as a means of increasing grain yield in maize has affected changes in leaf angle and shape as adaptations to this environment and has in general resulted in increased plant and ear heights. The stalk becomes mechanically weaker with increasing planting density because of reduction in individual plant vigor that results from a nonlinear relationship between planting density and biomass increase. (DeLoughery and Crookston (1979) Agronomy Journal, Vol. 71, July-August: 577-580)

The current disclosure also provides plants with increased leaf appearance rate, faster senescence, earlier days to shed and earlier days to silk, when compared to control plants, In one aspect, these plants can be useful for producing early maturing varieties.

The methods and compositions disclosed herein provide plants with altered architecture that could be useful for increasing planting densities.

The methods and compositions disclosed herein provide plants with reduced grain moisture,

In one aspect, the corn plants described herein are planted at a planting density of about 20,000 plants to about 50,000 plants per acre.

In one aspect, the methods of the invention find use in producing dwarf varieties of crop plants.

Dwarf crop plants having improved agronomic characteristics, such as, for example, reduced efficiency and increased yield per unit area are obtained by these methods.

By “dwarf” is intended to mean atypically small. By “dwarf plant” is intended to mean an atypically small plant. Generally, such a “dwarf plant” has a stature or height that is reduced from that of a typical plant by about 5%, 10%, 15%, 20%, 25%, 30%, 35%, 40%, 45%, 50%, 55%, 60% or greater. Generally, but not exclusively, such a dwarf plant is characterized by a reduced stem, stalk or trunk length when compared to the typical plant.

“Stay-green” or “staygreen” is a term used to describe a plant phenotype, e.g., whereby leaf senescence (most easily distinguished by yellowing of leaf associated with chlorophyll degradation) is delayed compared to a standard reference or a control.

“Photoperiodism” as used herein is defined as the response or capacity of plants to respond to photoperiod.

“Photoperiod” is defined as a daily recurring pattern of dark and light periods.

“Hypersensitivity” or “enhanced response” of a plant to day length of the day means that the plant exhibits alteration in an agronomic characteristic such as flowering time, or leaf appearance rate, or exhibits increased magnitude of response than the control plant when subjected to shorter or longer days than the critical day length.

“Transgenic” generally refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” generally refers to the RNA that is without introns and that can be translated into protein by the cell.

“cDNA” generally refers to a DNA that is complementary to and synthesized from a mRNA template using the enzyme reverse transcriptase. The cDNA can be single-stranded or converted into the double-stranded form using the Klenow fragment of DNA polymerase I.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein generally refers to a post-translationally processed polypeptide; i.e., one from which any pre- or pro-peptides present in the primary translation product have been removed.

“Precursor” protein generally refers to the primary product of translation of mRNA; i.e., with pre- and pro-peptides still present. Pre- and pro-peptides may be and are not limited to intracellular localization signals.

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

As used herein the terms non-genomic nucleic acid sequence or non-genomic nucleic acid molecule generally refer to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with a genomic nucleic acid sequence; insertion of one or more heterologous introns; deletion of one or more upstream or downstream regulatory regions associated with a genomic nucleic acid sequence; insertion of one or more heterologous upstream or downstream regulatory regions; deletion of the 5′ and/or 3′ untranslated region associated with a genomic nucleic acid sequence; and insertion of a heterologous 5′ and/or 3′ untranslated region.

“Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” generally refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” generally refers to a promoter whose activity is determined by developmental events.

“Operably linked” generally refers to the association of nucleic acid fragments in a single fragment so that the function of one is regulated by the other. For example, a promoter is operably linked with a nucleic acid fragment when it is capable of regulating the transcription of that nucleic acid fragment.

“Expression” generally refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” generally refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made (Lee et al. (2008) Plant Cell 20:1603-1622). The terms “chloroplast transit peptide” and “plastid transit peptide” are used interchangeably herein. “Chloroplast transit sequence” generally refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino acid sequence which directs a precursor protein into the mitochondria (Zhang and Glaser (2002) Trends Plant Sci 7:14-21).

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Complete sequences and figures for vectors described herein (e.g., pHSbarENDs2, pDONR™/Zeo, pDONRTM221, pBC-yellow, PHP27840, PHP23236, PHP10523, PHP23235 and PHP28647) are given in PCT Publication No. WO/2012/058528, the contents of which are herein incorporated by reference.

Turning now to the embodiments:

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

Isolated Polynucleotides and Polypeptides:

The present disclosure includes the following isolated polynucleotides and polypeptides:

An isolated polynucleotide comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 6.4%, 65%, 66%, 67%, 68%, 69%, 70%, 71%, 72%, 73%, 74.%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 13, 14, 15, 16, 17, 18 or 20, and combinations thereof; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs (including suppression DNA constructs) of the present disclosure. The polypeptide is a MATE-efflux polypeptide.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO: 2, 4, 6, 8, 10, 12, 13, 14, 15, 16, 17, 18 or 20, and combinations thereof. The polypeptide is a MATE-efflux polypeptide.

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1, 3, 5, 7, 9 or 19, and combinations thereof; or (ii) a full complement of the nucleic acid sequence of (i). Any of the foregoing isolated polynucleotides may be utilized in any recombinant DNA constructs (including suppression DNA constructs) of the present disclosure. The isolated polynucleotide encodes a MATE-efflux polypeptide.

An isolated polynucleotide comprising a nucleotide sequence, wherein the nucleotide sequence is hybridizable under stringent conditions with a DNA molecule comprising the full complement of SEQ ID NO:1, 3, 5, 7, 9 or 19. The isolated polynucleotide encodes a MATE-efflux polypeptide.

An isolated polynucleotide comprising a nucleotide sequence, wherein the nucleotide sequence is derived from SEQ ID NO:1, 3, 5, 7, 9 or 19 by alteration of one or more nucleotides by at least one method selected from the group consisting of: deletion, substitution, addition and insertion. The isolated polynucleotide encodes a MATE-efflux polypeptide.

An isolated polynucleotide comprising a nucleotide sequence, wherein the nucleotide sequence corresponds to an allele of SEQ ID NO:1, 3, 5, 7, 9 or 19.

It is understood, as those skilled in the art will appreciate, that the disclosure encompasses more than the specific exemplary sequences.

Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current disclosure may also be a protein which comprises an amino acid sequence comprising deletion, substitution, insertion and/or addition of one or more amino acids in an amino acid sequence presented in SEQ ID NO:2, 4, 6, 8, 10, 12, 13, 14, 15, 16, 17, 18 or 20. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present disclosure may also be a protein which is encoded by a nucleic acid comprising a nucleotide sequence comprising deletion, substitution, insertion and/or addition of one or more nucleotides in the nucleotide sequence of SEQ ID NO:1, 3, 5, 7, 9 or 19. Nucleotide deletion, substitution, insertion and/or addition may be accomplished by site-directed mutagenesis or other techniques as mentioned above.

The protein of the present disclosure may also be a protein which is encoded by a nucleic acid comprising a nucleotide sequence hybridizable under stringent conditions with the complementary strand of the nucleotide sequence of SEQ ID NO:1, 3, 5, 7, 9 or 19.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

Recombinant DNA Constructs and Suppression DNA Constructs:

In one aspect, the present disclosure includes recombinant DNA constructs (including suppression DNA constructs).

In one aspect, a recombinant DNA construct comprises a polynucleotide operably linked to at least one heterologous regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 13, 14, 15, 16, 17, 18 or 20, and combinations thereof; or (ii) a full complement of the nucleic acid sequence of (i).

In another embodiment, a recombinant DNA construct comprises a polynucleotide operably linked to at least one heterologous regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1, 3, 5, 7, 9 or 19, and combinations thereof; or (ii) a full complement of the nucleic acid sequence of (i).

In one embodiment, a recombinant DNA construct comprises a polynucleotide operably linked to at least one heterologous regulatory sequence (e.g., a promoter functional in a plant), wherein the polynucleotide encodes a MATE-efflux polypeptide, and wherein said recombinant DNA construct confers upon a plant comprising said recombinant DNA construct at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, earlier maturity and reduced grain moisture, when compared to a control plant not comprising said recombinant DNA construct.

The MATE-efflux polypeptide may be from Arabidopsis thaliana, Zea mays, Glycine max, Glycine tabacina, Glycine soja, Glycine tomentella, Oryza sativa, Brassica napus, Sorghum bicolor, Saccharum officinarum, or Triticum aestivum.

In another aspect, the present disclosure includes suppression DNA constructs.

A suppression DNA construct may comprise at least one heterologous regulatory sequence (e.g., a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 13, 14, 15, 16, 17, 18 or 20, and combinations thereof, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a MATE-efflux polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1, 3, 5, 7, 9 or 19, and combinations thereof, or (ii) a full complement of the nucleic acid sequence of (c)(i). The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an siRNA construct or an miRNA construct).

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

A suppression DNA construct may comprise 100, 200, 300, 400, 500, 600, 700, 800, 900 or 1000 contiguous nucleotides of the sense strand (or antisense strand) of the gene of interest, and combinations thereof.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

Suppression of gene expression may also be achieved by use of artificial miRNA precursors, ribozyme constructs and gene disruption. A modified plant miRNA precursor may be used, wherein the precursor has been modified to replace the miRNA encoding region with a sequence designed to produce a miRNA directed to the nucleotide sequence of interest. Gene disruption may be achieved by use of transposable elements or by use of chemical agents that cause site-specific mutations.

“Antisense inhibition” generally refers to the production of antisense RNA transcripts capable of suppressing the expression of the target gene or gene product. “Antisense RNA” generally refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” generally refers to the production of sense RNA transcripts capable of suppressing the expression of the target gene or gene product. “Sense” RNA generally refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference generally refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

The terms “miRNA-star sequence” and “miRNA* sequence” are used interchangeably herein and they refer to a sequence in the miRNA precursor that is highly complementary to the miRNA sequence. The miRNA and miRNA* sequences form part of the stem region of the miRNA precursor hairpin structure.

In one embodiment, there is provided a method for the suppression of a target sequence comprising introducing into a cell a nucleic acid construct encoding a miRNA substantially complementary to the target. In some embodiments the miRNA comprises about 19, 20, 21, 22, 23, 24 or 25 nucleotides. In some embodiments the miRNA comprises 21 nucleotides. In some embodiments the nucleic acid construct encodes the miRNA. In some embodiments the nucleic acid construct encodes a polynucleotide precursor which may form a double-stranded RNA, or hairpin structure comprising the miRNA.

In some embodiments, the nucleic acid construct comprises a modified endogenous plant miRNA precursor, wherein the precursor has been modified to replace the endogenous miRNA encoding region with a sequence designed to produce a miRNA directed to the target sequence. The plant miRNA precursor may be full-length of may comprise a fragment of the full-length precursor. In some embodiments, the endogenous plant miRNA precursor is from a dicot or a monocot. In some embodiments the endogenous miRNA precursor is from Arabidopsis, tomato, maize, soybean, sunflower, sorghum, canola, wheat, alfalfa, cotton, rice, barley, millet, sugar cane or switchgrass.

In some embodiments, the miRNA template, (i.e. the polynucleotide encoding the miRNA), and thereby the miRNA, may comprise some mismatches relative to the target sequence. In some embodiments the miRNA template has >1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 9.4%, 95%, 96%, 97%, 98%, 99%, or 100% as compared to the complement of the target sequence.

In some embodiments, the miRNA template, (i.e. the polynucleotide encoding the miRNA) and thereby the miRNA, may comprise some mismatches relative to the miRNA-star sequence. In some embodiments the miRNA template has >1 nucleotide mismatch as compared to the miRNA-star sequence, for example, the miRNA template can have 1, 2, 3, 4, 5, or more mismatches as compared to the miRNA-star sequence. This degree of mismatch may also be described by determining the percent identity of the miRNA template to the complement of the miRNA-star sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA-star sequence.

Regulatory Sequences:

A recombinant DNA construct (including a suppression DNA construct) of the present disclosure may comprise at least one regulatory sequence.

A regulatory sequence may be a promoter.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance stress tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)). Endosperm preferred promoters include those described in e.g., U.S. Pat. No. 8,466,342; U.S. Pat. No. 7,897,841; and U.S. Pat. No. 7,847,160.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Promoters for use also include the following: Zm-GOS2 (maize promoter for “Gene from Oryza sativa”, US publication number US2012/0110700 Sb-RCC (Sorghum promoter for Root Cortical Cell delineating protein, root specific expression), Zm-ADF4 (U.S. Pat. No. 7,902,428; Maize promoter for Actin Depolymerizing Factor), Zm-FTM1 (U.S. Pat. No. 7,842,851; maize promoter for Floral transition MADSs) promoters.

Additional promoters for regulating the expression of the nucleotide sequences in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In one embodiment the at least one regulatory element may be an endogenous promoter operably linked to at least one enhancer element; e.g., a 35S, nos or ocs enhancer element.

Promoters for use may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1 BIO promoter (WO06055487, published May 26, 2006), the CRWAQ81 (WO05035770, published Apr. 21, 2005) and the maize ZRP2.47 promoter (NCBI accession number: U38790; GI No. 1063664),

Recombinant DNA constructs of the present disclosure may also include other regulatory sequences, including but not limited to, translation leader sequences, introns, and polyadenylation recognition sequences. In another embodiment of the present disclosure, a recombinant DNA construct of the present disclosure further comprises an enhancer or silencer.

The promoters disclosed herein may be used with their own introns, or with any heterologous introns to drive expression of the transgene.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

Improper termination of an RNA transcript can affect the stability of the RNA, and hence can affect protein expression. Variability of transgene expression is sometimes attributed to variability of termination efficiency (Bieri et al (2002) Molecular Breeding 10: 107-117).

Examples of terminators for use include, but are not limited to, PinII terminator, SB-GKAF terminator (U.S. Appln. No. 61/514,055), Actin terminator, Os-Actin terminator, Ubi terminator, Sb-Ubi terminator, Os-Ubi terminator.

Any plant can be selected for the identification of regulatory sequences and MATE-efflux polypeptide genes to be used in recombinant DNA constructs and other compositions (e.g. transgenic plants, seeds and cells) and methods of the present disclosure. Examples of suitable plants for the isolation of genes and regulatory sequences and for compositions and methods of the present disclosure would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

Compositions:

A composition of the present disclosure includes a transgenic microorganism, cell, plant, and seed comprising the recombinant DNA construct. The cell may be eukaryotic, e.g., a yeast, insect or plant cell, or prokaryotic, e.g., a bacterial cell.

A composition of the present disclosure is a plant comprising in its genome any of the recombinant DNA constructs (including any of the suppression DNA constructs) of the present disclosure (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., shorter plant stature, reduced days to shed, earlier flowering, reduced days to silk, earlier senescence, shorter life cycle, increased leaf number, reduced stalk diameter, hypersensitivity to day length, and reduced grain moisture, faster leaf appearance rate, earlier maturity), or used in a breeding program to produce hybrid seed, which can be grown to produce plants that would exhibit such an altered agronomic characteristic. The seeds may be maize seeds. The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

The recombinant DNA construct may be stably integrated into the genome of the plant.

Particular embodiments include but are not limited to the following:

1. A plant (for example, a maize, rice or soybean plant) comprising in its genome a recombinant DNA construct comprising a polynucleotide operably linked to at least one heterologous regulatory element, wherein said polynucleotide encodes a MATE-efflux polypeptide, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising said recombinant DNA construct.

2. A plant (for example, a maize, rice or soybean plant) comprising in its genome a recombinant DNA construct comprising a polynucleotide operably linked to at least one regulatory sequence, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 13, 14, 15, 16, 17, 18 or 20, and wherein said plant exhibits at least one altered agronomic characteristic selected from the group consisting of shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising said recombinant DNA construct.

3. A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:1, 3, 5, 7, 9 or 19 by alteration of one or more nucleotides by at least one method selected from the group consisting of: deletion, substitution, addition and insertion; and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising said recombinant DNA construct.

4. One embodiment is a plant comprising in its genome a recombinant DNA construct comprising a polynucleotide operably linked to at least one heterologous regulatory element, wherein said polynucleotide encodes a MATE-efflux polypeptide, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising said recombinant DNA construct, wherein the polynucleotide comprises a nucleotide sequence that has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1, 3, 5, 7, 9 or 19, and wherein the polynucleotide sequence can be modified by Cas9 nuclease/guide-RNA mediated genome editing approach.

5. Another embodiment is a plant comprising in its genome an endogenous polynucleotide operably linked to at least one heterologous regulatory element, wherein said endogenous polynucleotide encodes a MATE-efflux polypeptide having an amino acid sequence that has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 13, 14, 15, 16, 17, 18 or 20, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising the heterologous regulatory element operably linked to the endogenous polynucleotide. In one embodiment, the at least one heterologous regulatory element is at least one regulatory element endogenous to the plant.

6. In one embodiment, the plant disclosed herein is a monocot plant. In one embodiment, the monocot plant is a maize plant.

7. A plant (for example, a maize, rice or soybean plant) comprising in its genome a recombinant DNA construct comprising a polynucleotide operably linked to at least one regulatory element, wherein said polynucleotide encodes a polypeptide having an amino acid sequence of at least 50%, 51%, 52%, 53%, 5.4%, 55%, 56%, 57%, 58%, 59%, 60%, 61%, 62%, 63%, 6.4%, 65%, 66%, 67%, 68%, 69%, 70%, 71%, 72%, 73%, 74.%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 13, 14, 15, 16, 17, 18 or 20, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising said recombinant DNA construct.

8. A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:1, 3, 5, 7, 9 or 19 by alteration of one or more nucleotides by at least one method selected from the group consisting of: deletion, substitution, addition and insertion; and wherein said plant exhibits an alteration of at least one agronomic characteristic when compared to a control plant not comprising said recombinant DNA construct.

9. A plant (for example, a maize, rice or soybean plant) comprising in its genome a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a MATE-efflux polypeptide, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising said suppression DNA construct.

10. A plant (for example, a maize, rice or soybean plant) comprising in its genome a suppression DNA construct comprising at least one regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO: 2, 4, 6, 8, 10, 12, 13, 14, 15, 16, 17, 18 or 20, or (b) a full complement of the nucleic acid sequence of (a), and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising said suppression DNA construct.

11. A plant (for example, a maize, rice or soybean plant) comprising in its genome a polynucleotide (optionally an endogenous polynucleotide) operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 13, 14, 15, 16, 17, 18 or 20, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising the recombinant regulatory element. The at least one heterologous regulatory element may comprise an enhancer sequence or a multimer of identical or different enhancer sequences. The at least one heterologous regulatory element may comprise one, two, three or four copies of the CaMV 35S enhancer.

12. Any progeny of the plants in the embodiments described herein, any seeds of the plants in the embodiments described herein, any seeds of progeny of the plants in embodiments described herein, and cells from any of the above plants in embodiments described herein and progeny thereof.

In an embodiment, the plants disclosed herein exhibit modified plant architecture or change in harvest index. In one aspect, the modified plant architecture includes a modification selected from the group consisting of increased harvest index, shorter stature, reduced leaf angle, and reduced canopy.

In one aspect, any of the plants disclosed herein exhibits faster leaf appearance rate and earlier maturity.

In one aspect, the relative maturity of corn is reduced by modulating a maturity parameter selected from the group consisting of flowering time, grain filling and senescence.

In any methods and/or compositions described herein, the MATE-efflux polypeptide may be from Arabidopsis thaliana, Zea mays, Glycine max, Glycine tabacina, Glycine soja, Glycine tomentella, Oryza sativa, Brassica napus, Sorghum bicolor, Saccharum officinarum, or Triticum aestivum.

In any of the aspects of the current disclosure, the recombinant DNA construct (or suppression DNA construct) may comprise at least a promoter functional in a plant as a regulatory sequence.

In any of the aspects described herein or any other aspects of the present disclosure, the alteration of at least one agronomic characteristic is either an increase or decrease.

In any of the aspects described herein, the at least one agronomic characteristic may be selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant stature or height, ear height, ear length, salt tolerance, reduced days to shed, earlier flowering, reduced days to silk, earlier senescence, shorter life cycle, earlier maturity, increased leaf number, reduced stalk diameter, hypersensitivity to day length, and reduced grain moisture, early seedling vigor and seedling emergence under low temperature stress.

In any of the aspects described herein, the plant may exhibit the alteration of at least one agronomic characteristic when compared, under stress or non-stress conditions, to a control plant not comprising said recombinant DNA construct (or said suppression DNA construct). The at least one stress condition may be an abiotic stress.

In any of the aspects described herein, the plant may be planted at a planting density of about 20,000 plants to about 50,000 plants per acre.

For example, planting densities of about 15,000, 18,000, 22,000, 24,000, 25,000, 28,000, 30,000, 32,000, 34,000, 36,000, 38,000, 40,000 and 42,000 may be used. The row width range can include 30-inch rows, 24-inch rows, 20-inch rows, 18-inch rows or narrower. The reduced stature of the corn plants disclosed herein is advantageous for narrower row spacing, thereby increasing the planting density.

In one aspect, the plants disclosed herein do not exhibit an agronomic penalty. In one aspect, the plants disclosed herein do not exhibit an agronomic penalty such as yield penalty.

One of ordinary skill in the art would readily recognize a suitable control or reference plant to be utilized when assessing or measuring an agronomic characteristic or phenotype of a transgenic plant in any embodiment of the present disclosure in which a control plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or the suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Methods:

Methods include but are not limited to methods for conferring upon a plant at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising increasing the expression of a MATE-efflux protein in the plant, methods for determining an alteration of an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Methods include but are not limited to the following:

A method for transforming a cell (or microorganism) comprising transforming a cell (or microorganism) with any of the isolated polynucleotides or recombinant DNA constructs of the present disclosure. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or recombinant DNA constructs (including suppression DNA constructs) of the present disclosure and regenerating a transgenic plant from the transformed plant cell. The disclosure is also directed to the transgenic plant produced by this method, and transgenic seed obtained from this transgenic plant. The transgenic plant obtained by this method may be used in other methods of the present disclosure.

A method for isolating a polypeptide of the disclosure from a cell or culture medium of the cell, wherein the cell comprises a recombinant DNA construct comprising a polynucleotide of the disclosure operably linked to at least one regulatory sequence, and wherein the transformed host cell is grown under conditions that are suitable for expression of the recombinant DNA construct.

A method of altering the level of expression of a polypeptide of the disclosure in a host cell comprising: (a) transforming a host cell with a recombinant DNA construct of the present disclosure; and (b) growing the transformed host cell under conditions that are suitable for expression of the recombinant DNA construct wherein expression of the recombinant DNA construct results in production of altered levels of the polypeptide of the disclosure in the transformed host cell.

A method of conferring upon a plant at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, earlier maturity, faster leaf appearance rate, and reduced grain moisture, the method comprising increasing the expression of a MATE-efflux protein in the plant.

A method of conferring upon a plant at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising the steps of (a) introducing into a regenerable plant cell a recombinant DNA construct comprising a polynucleotide operably linked to at least one heterologous regulatory sequence, wherein the polynucleotide encodes a MATE-efflux polypeptide having an amino acid sequence that has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity sequence identity, based on Clustal V or Clustal W method of alignment, when compared to SEQ ID NO:2, 4, 6, 8, 10, 12, 13, 14, 15, 16, 17, 18 or 20; (b) regenerating a transgenic plant from the regenerable plant cell of (a), wherein the transgenic plant comprises in its genome the recombinant DNA construct.

The method may further comprise (c) obtaining a progeny plant derived from the transgenic plant of (b), wherein said progeny plant comprises in its genome the recombinant DNA construct and exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the recombinant DNA construct.

A method of conferring upon a plant at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising the steps of (a) introducing into a regenerable plant cell a recombinant DNA construct comprising a polynucleotide operably linked to at least one heterologous regulatory sequence, wherein said polynucleotide comprises a nucleotide sequence, wherein the nucleotide sequence is: (a) hybridizable under stringent conditions with a DNA molecule comprising the full complement of SEQ ID NO:1, 3, 5, 7, 9 or 19; or (b) derived from SEQ ID NO:1, 3, 5, 7, 9 or 19, by alteration of one or more nucleotides by at least one method selected from the group consisting of: deletion, substitution, addition and insertion; and (b) regenerating a transgenic plant from the regenerable plant cell after step (a), wherein the transgenic plant comprises in its genome the recombinant DNA construct and exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the recombinant DNA construct. The method may further comprise (c) obtaining a progeny plant derived from the transgenic plant, wherein said progeny plant comprises in its genome the recombinant DNA construct and exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the recombinant DNA construct.

A method of selecting for (or identifying) a plant that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising: (a) obtaining a transgenic plant, wherein the transgenic plant comprises in its genome a recombinant DNA construct comprising a polynucleotide operably linked to at least one heterologous regulatory element, wherein said polynucleotide encodes a MATE-efflux polypeptide having an amino acid sequence that has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2, 4, 6, 8, 10, 12, 13, 14, 15, 16, 17, 18 or 20; (b) growing the transgenic plant of part (a) under conditions wherein the polynucleotide is expressed; and (c) selecting the transgenic plant of part (b) with at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the recombinant DNA construct.

A method of selecting for (or identifying) a plant that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising: (a) obtaining a transgenic plant, wherein the transgenic plant comprises in its genome a recombinant DNA construct comprising a polynucleotide operably linked to at least one regulatory element, wherein said polynucleotide comprises a nucleotide sequence, wherein the nucleotide sequence is: (i) hybridizable under stringent conditions with a DNA molecule comprising the full complement of SEQ ID NO:1, 3, 5, 7, 9 or 19; or (ii) derived from SEQ ID NO:1, 3, 5, 7, 9 or 19 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting the transgenic plant of part (b) with at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the recombinant DNA construct.

One aspect of the current disclosure is a method of increasing yield of a crop plant, the method comprising increasing expression of a MATE-efflux protein in the crop plant. In one embodiment, the crop plant is planted at a density higher than a control crop plant.

One aspect is method of selecting a plant that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising the steps of: (a) introducing a mutation into an endogenous MATE-efflux gene of a plant, to create a mutant plant comprising a MATE-efflux mutant gene; and (b) selecting the mutant plant of step (a) that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the MATE-efflux mutant gene. In one embodiment, step (a) is done using at least one method selected from the group consisting of: Targeting Induced Local Lesions IN Genomics (TILLING), transposon tagging, and CRISPR technology. In one aspect, the mutation is in a non-coding region of the MATE-efflux gene.

A method of producing seed (for example, seed that can be sold as a drought tolerant product offering) comprising any of the preceding methods, and further comprising obtaining seeds from said progeny plant, wherein said seeds comprise in their genome said recombinant DNA construct (or suppression DNA construct).

In any of the preceding methods or any other aspects of methods of the present disclosure, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other aspects of methods of the present disclosure, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other aspects of methods of the present disclosure, the at least one agronomic characteristic may be selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant stature or height, ear height, ear length, salt tolerance, reduced days to shed, earlier flowering, reduced days to silk, earlier senescence, shorter life cycle, increased leaf number, reduced stalk diameter, hypersensitivity to day length, earlier maturity, faster leaf appearance rate, and reduced grain moisture, early seedling vigor and seedling emergence under low temperature stress.

The alteration may be an increase or decrease.

In any of the preceding methods or any other aspects of methods of the present disclosure, the plant may exhibit the alteration of at least one agronomic characteristic when compared, under stress conditions, wherein the stress is selected from the group consisting of drought stress, triple stress and osmotic stress, to a control plant not comprising said recombinant DNA construct (or said suppression DNA construct).

In any of the preceding methods or any other aspects of methods of the present disclosure, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of recombinant DNA constructs of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines.

Conversely, pollen from plants of these important lines is used to pollinate regenerated plants. A transgenic plant of the present disclosure containing a desired polypeptide is cultivated using methods well known to one skilled in the art.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Mutant Identification and Isolation of MATE9 Gene

A T-DNA insertion event in maize plants was identified to show an early flowering phenotype, when homozygous plants from the event were planted in a field observation experiment in a non-stress location. Flowering time and plant height data were collected from single row plots in 3 replicates. The silk dates for plants from this event were about 8 days earlier than wild type plants, whereas shed dates were about 4 days earlier. At the same time, plant height was reduced by about 30%. This early flowering and reduced stature phenotype was only observed in the homozygous plants from a single event, indicating it's a recessive trait unrelated to the original transgene and likely caused by disruption of a different gene. This was confirmed through mapping of the transgene insertion site.

Mapping the transgene insertion site led to the discovery of maize MATE9 gene (CDS given in SEQ ID NO:1), as the transgene was found inserting in the only intron of MATE9. Genomic DNA from the homozygous plants was isolated. By PCR using primers designed against the T-DNA borders, and fully sequencing the junctions between genomic DNA and T-DNA borders, the transgene insertion was found to contain the full and intact T-DNA carrying an un-related transgene. The same transgene was also in a number of other events, none of them had a flowering or stature phenotype. The insertion mutant event also did not show any phenotype in a heterozygous background.

### Example 2

### Evaluating Day-Length Responses in Plants Under Controlled Environment

Two growth chambers were set up to provide either long day (16 hour light, 8 hour dark) or short day (10 hour light, 14 hour dark). MATE9 mutant as well as wildtype plants were grown under these conditions until flowering, with 12 plants per genotype per chamber. Leaf counts were measured regularly as V-stages, the numbers of leaves with leaf collar (i.e., V4=4 collared leaves). The change in leaf counts over time can be expressed as leaf appearance rates, in this case, simply the number of days it takes to add a leaf.

The hypersensitivity of the MATE9 mutant plant to day length was measured as determined by the leaf appearance rate at stage V. Wild type plants were used as controls. Hypersensitivity under long day (16 hour) under short day (10 hour) were demonstrated for the MATE9 mutant.

### Example 3

### Evaluating Leaf Appearance Rates in Field-Grown Plants

Plants grown under field conditions were monitored for leaf counts over time, from emergence to flowering. At least 3 plants from each plot were included in the measurements, in 2 or 3 replicated plots. The corresponding calendar dates were then converted to heat units, based on daily temperature over that period of time. The change in leaf counts over time can be expressed as leaf appearance rates, in this case, more precisely as the amount of heat units per leaf produced.

The leaf appearance rate for the Zea mays MATE9 (SEQ ID NO:2) overexpression construct pUbi_ZmMATE9 (63.10) and the MATE9 RNAi construct pUbiZmMATE9_RNAi1 (77.14) were measured as compared to the control plant (77.22), for the full duration of time between leaf emergence to VT stage. The leaf appearance rate for the maize MATE9 (SEQ ID NO:2) overexpression construct pUbi_ZmMATE9 (69.99) and the MATE9 RNAi construct pUbiZmMATE9_RNAi1 (78.76), compared to the control plant (77.97), from emergence to V6 stage, was measured. The leaf appearance rate for the maize MATE9 (SEQ ID NO:2) overexpression construct pUbi_ZmMATE9 (57.80) and the MATE9 RNAi construct pUbiZmMATE9_RNAi1 (76.14), compared to the control plant (77.14), from V6 to VT stage, were measured.

### Example 4

### Evaluating Leaf Senescence in Field-Grown Plants

Plants overexpressing MATE9, and plants with suppression of MATE9 expression were grown under field conditions and were monitored for leaf greenness over time, from shortly after flowering to the end of season, or until plants were fully senesced with brown leaves. Leaf color was assessed visually across all plants of each plot. A stay-green score (STAGRN) was assigned based on the percentage of leaves that are green, for example, STAGRN=7.5 if 75% of all leaves are green, and STAGRN=3 if only 30% leaves are green. The scores were collected across 3 replicated plots.

Faster senescence in plants overexpressing MATE9 polypeptide (SEQ ID NO:2), compared to the rate of senescence in plants containing the MATE9 RNAi constructs pUbi_ZmMATE9_RNAi1 and pUbi_ZmMATE9_RNAi2, were observed.

### Example 5

### Evaluating Flowering Times in Plants Grown Under Greenhouse Conditions

Early flowering in a MATE7 (SEQ ID NO:4) overexpressing maize plant was assessed, wherein the experiment was done in fast corn under greenhouse conditions. A TO plant from one of the 10 events transformed with UBI::ZmMATE7 construct, was compared to a control plant that went through the transformation process without a construct. The plants were photographed 28 days after transplanting from tissue culture media and the MATE7 overexpressing plant had early flowering compared to the control plant.

### Example 6

### Evaluating Plant Stature in Plants Grown Under Greenhouse Conditions

Differences in plant stature between control plant and ZmMATE7 (SEQ ID NO:4) overexpressing plants was assessed, the experiment was done with fast corn under greenhouse conditions. TO plants from 10 events transformed with UBI::ZmMATE7 construct illustrated high penetrance of the early flowering phenotype. Plants that flower early have a reduced stature and tassels were visible. The two events not expressing the transgene did not show a phenotype, and were considered as a negative controls. The plants were photographed 28 days after transplanting from tissue culture media.

### Example 7

### Evaluating Development in Plants Overexpressing MATE9 and Mutant Plants

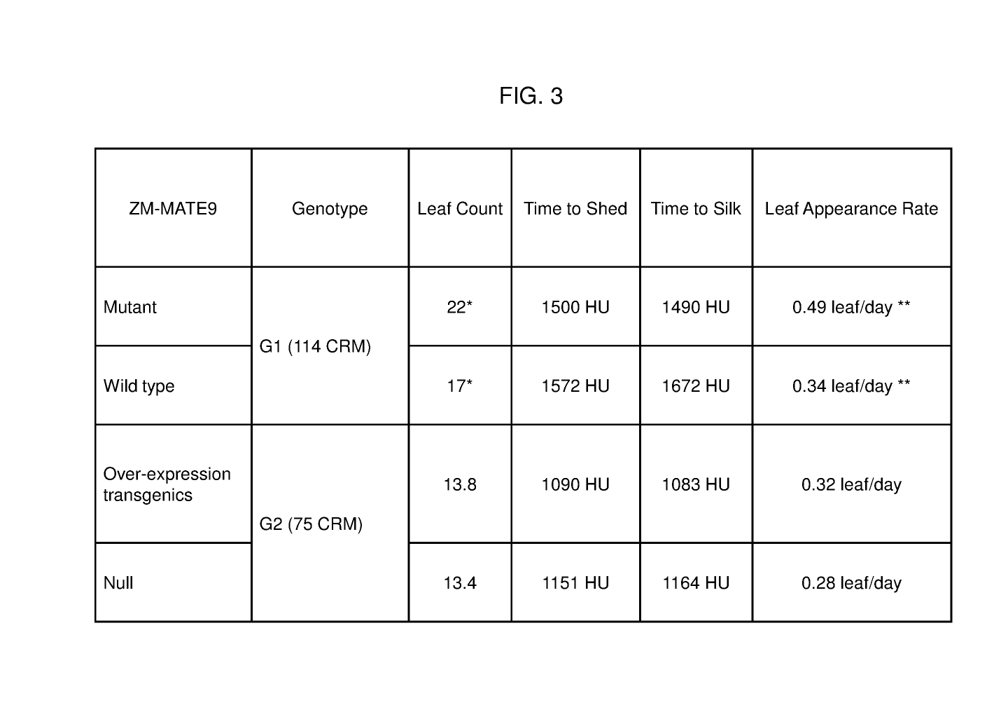

The effect on plant development in the original MATE9 mutant plant and in UBI::ZmMATE9 overexpressing maize plants was compared to wild-type and null transgenic plants (FIG. 3). Leaf count, time to shed (GDUSHD), time to silk (GDUSLK) and leaf appearance rate are compared. All data was collected from field conditions except for: the leaf count for the mutant and the wild-type plant, that was collected from plants grown in a greenhouse, indicated by a single asterisk; and the leaf appearance rate for the mutant and the wild-type plant, that was collected from plants grown in a 16-hr day growth chamber indicated by a double asterisk. CRM stands for corn relative maturity

### Example 8

### Characterization of cDNA Clones Encoding MATE-Efflux Polypeptides

cDNA libraries representing mRNAs from various tissues of Zea mays, were prepared and cDNA clones encoding MATE-efflux polypeptides were identified. The maize clones and MATE-efflux polypeptides identified from other plants are given in Table 1.

FIG. 1A-FIG. 1F show the alignment of the MATE-efflux polypeptides given in SEQ ID NOS:2, 4, 6, 8, 10-18 and 20. Residues that are identical to the residue of SEQ ID NO:2) at a given position are enclosed in a box. A consensus sequence (SEQ ID NO:21) is presented where a residue is shown if identical in all sequences, otherwise, a period is shown.

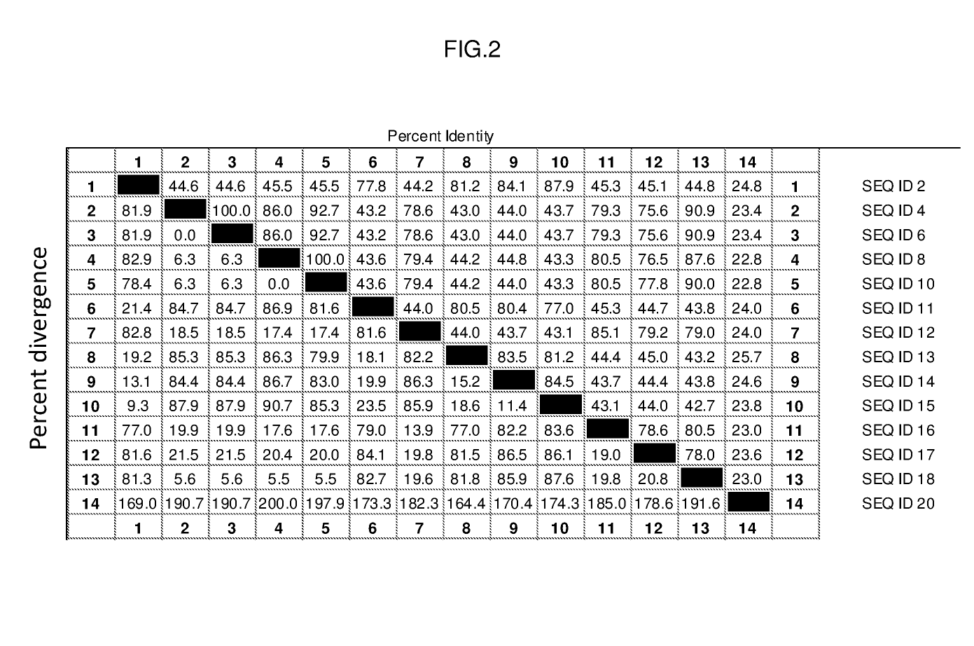

FIG. 2 shows the percent sequence identity and the divergence values for each pair of amino acids sequences of MATE efflux polypeptides displayed in FIG. 1A-1F.

Sequence alignments and percent identity calculations were performed using the Megalign® program of the LASERGENE® bioinformatics computing suite (DNASTAR® Inc., Madison, Wis.). Multiple alignment of the sequences was performed using the Clustal V method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE=1, GAP PENALTY=3, WINDOW=5 and DIAGONALS SAVED=5.

Sequence alignments and BLAST scores and probabilities indicate that the nucleic acid fragments comprising the instant cDNA clones encode DTP4 polypeptides.

### Example 9A

### Analysis of Maize Lines with the MATE Gene for Yield and Other Agronomic Characteristics

A recombinant DNA construct containing a MATE-efflux gene can be introduced into an elite maize inbred line either by direct transformation or introgression from a separately transformed line.

Transgenic plants, either inbred or hybrid, can undergo more vigorous field-based experiments to study agronomic traits such as ear height, plant height, grain moisture, GDU to shed, GDU to silk, leaf numbers and yield, under non-stress and stress conditions. Stress can be abiotic stress such as drought stress or nitrogen stress.

Subsequent analysis can be done to determine whether plants that contain the MATE-efflux gene have an effect on agronomic traits such as ear height, plant height, grain moisture, GDU to shed, GDU to silk, leaf number and yield performance, when compared to the control plants that do not overexpress the MATE efflux gene. Specifically, the MATE-efflux gene can be introduced into inbred lines of different maturity ratings, and transgenic plants planted in geographic regions corresponding to where those inbred lines, as well as related top-cross hybrids, are adapted to grow. The above method may be used to select transgenic plants with agronomic traits such as reduced GDU to shed and to silk, decreased grain moisture, alteration in plant stature, when compared to a control plant not comprising said recombinant DNA construct.

### Example 9B

### Field Analysis of Maize Lines Transformed with pUbi ZmMATE9 Encoding the Maize MATE9 Gene

The Zm-MATE9 polypeptide (SEQ ID NO:2) encoded by the nucleotide sequence (SEQ ID NO:1) present in the vector pUbi-ZmMATE9 was introduced into a transformable maize line derived from an elite maize inbred line. Maturity of the plants overexpressing the pUbi-ZmMATE9 was evaluated.

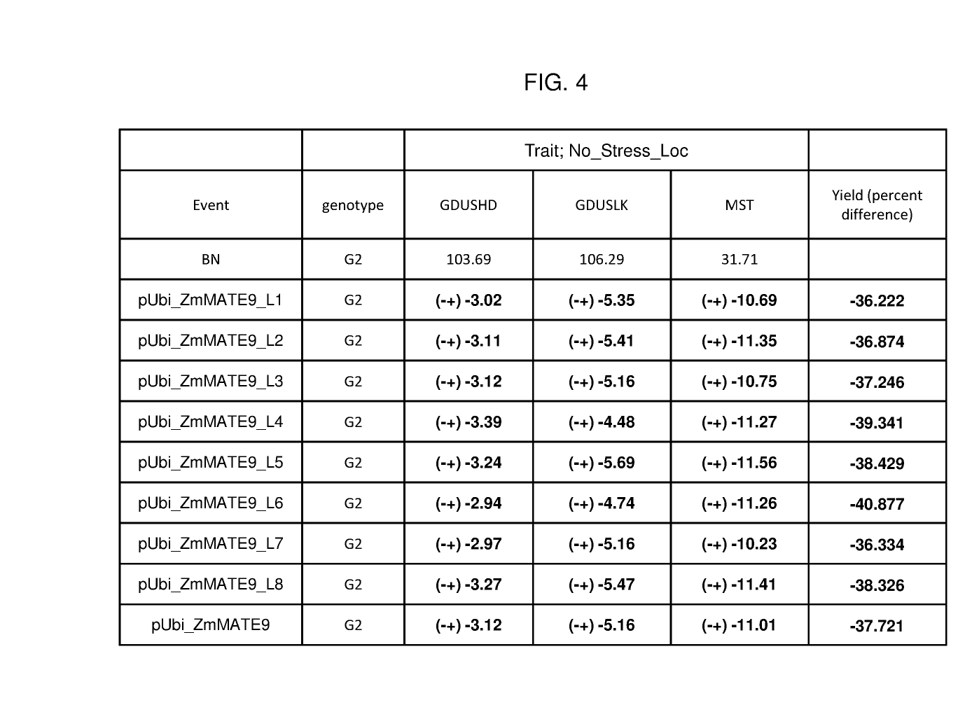

Eight transgenic events were field tested at 1 non stress location, No_Stress_Loc, as shown in FIG. 4. Data for shed time (GDUSHD), silking time (GDUSLK) and grain moisture content (MST) was collected, and is shown in FIG. 4. As FIG. 4 shows all the events showed reduced time to shed, reduced time to silk, and decreased moisture content (The significant values (with p-value less than or equal to 0.1 with a 2-tailed test) are shown in bold). The transgenic events overexpressing the ZM-MATE9 protein (SEQ ID NO:2) were almost 11 points lower than the Bulk null control plants in moisture content. The last row in FIG. 4 shows the construct average for all these three traits. Evaluation of these traits show that these transgenic plants reach maturity earlier than control plants.

Yield data (percent difference) for the 8 transgenic events is shown in the last column of FIG. 9. The planting density was 36,000 plants per acre. Yield analysis was by ASREML (VSN International Ltd), and the values shown are percent differences from the bulk null values. (Cullis, B. R et al (1998) Biometrics 54: 1-18, Gilmour, A. R. et al (2009). ASRemI User Guide 3.0, Gilmour, A. R., et al (1995) Biometrics 51: 1440-50).

As shown in FIG. 4, last column, negative effect of the transgene on yield was seen for all the events. Yield is shown as percent difference from the bulk null (difference of event yield from the bulk null/bulk null yield×100)

### Example 9C

### Field Analysis of Maize Lines Transformed with pUbi AtMATE EP1 Encoding the Arabidopsis Gene At1G61890

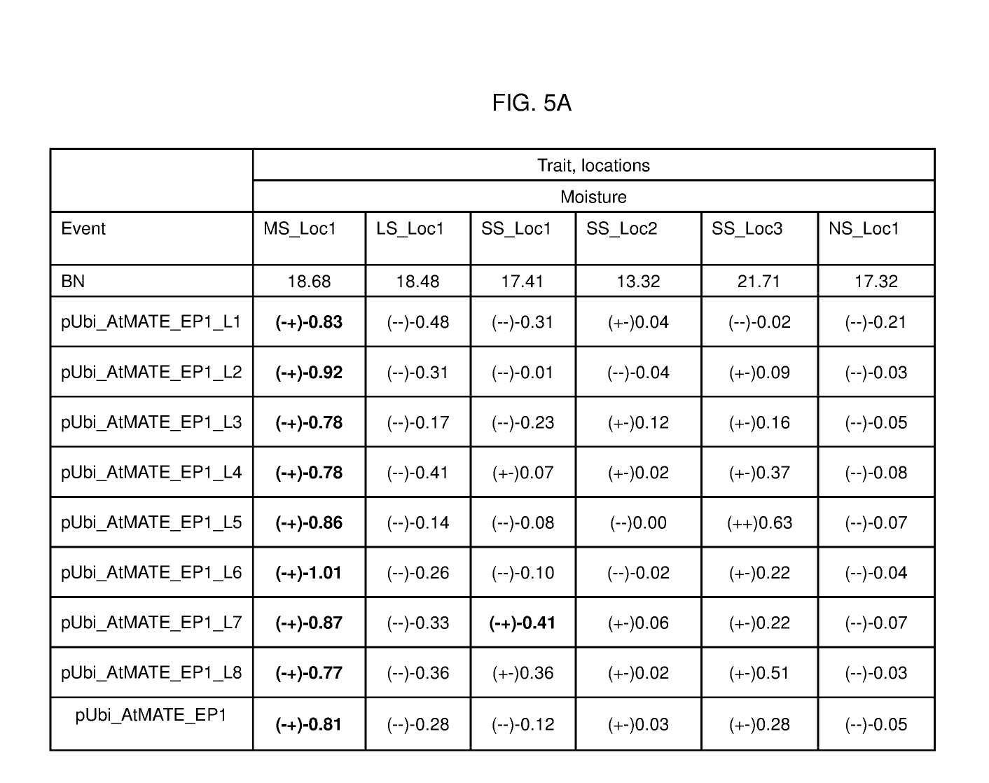

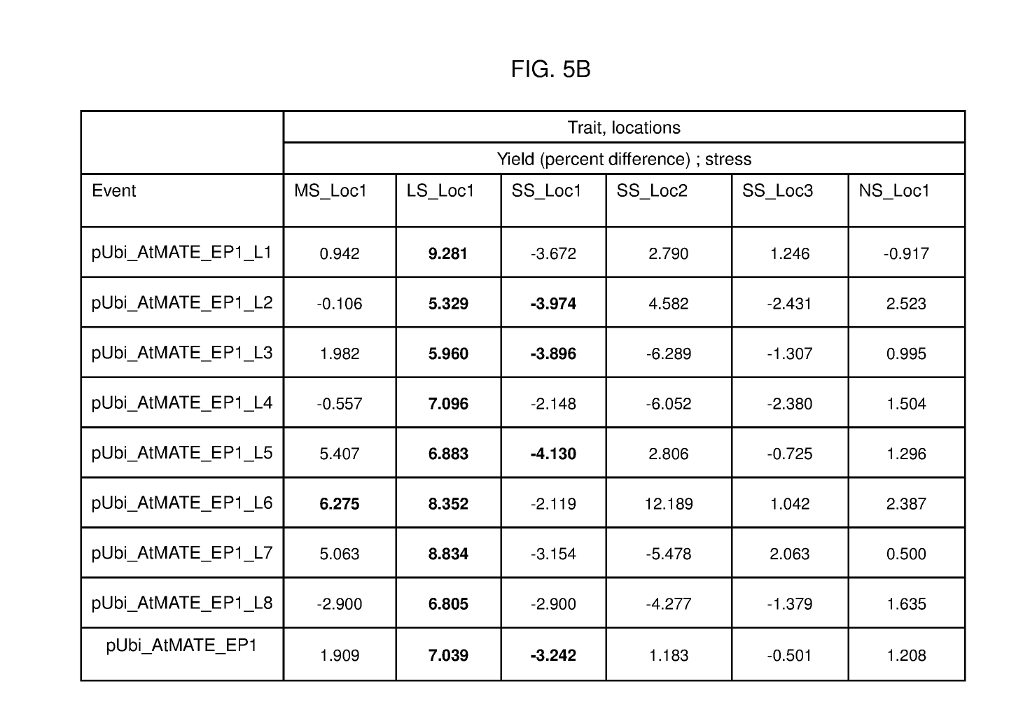

Eight transgenic events for the construct pUbi_AtMATE_EP1, that overexpressed the Arabidopsis MATE-efflux polypeptide (SEQ ID NO:20; encoded by the SEQ ID NO:19) were field tested at 6 locations, that included one medium stress location (MS_Loc1), one low stress location (LS_Loc1), three severe stress locations (SS_Loc1-3) and one non-stress location (NS_Loc1); shown in FIG. 5A. Data for grain moisture content was collected at the time of harvest (moisture data were collected in a combine automatically as grain is harvested) and compared with bulk null. Statistical significance is reported at P<0.1 for a two-tailed test.

The significant values (with p-value less than or equal to 0.1 with a 2-tailed test) are shown in bold.

As shown in FIG. 5A, in the medium stress location, MS_Loc1, the grain moisture was less in all the eight transgenic events, and one event pUbi_AtMATE_EP1-L7_showed less moisture content in one severe stress location.

Yield analysis for pUbi_AtMATE_EP1 was done and yield data (percent difference) for the 8 transgenic events is shown in FIG. 5B. Yield analysis was by ASREML (VSN International Ltd), and the values shown are percent differences from the bulk null values. (Cullis, B. R et al (1998) Biometrics 54: 1-18, Gilmour, A. R. et al (2009). ASRemI User Guide 3.0, Gilmour, A. R., et al (1995) Biometrics 51: 1440-50).

As shown in FIG. 5B, positive effect of the transgene on yield was seen for all the events in one location. Yield is shown as percent difference from the bulk null (difference of event yield from the bulk null/bulk null yield×100)

### Example 9D

### Field Analysis of Maize Lines Transformed with pUbi-ZmMATE EP1 Encoding the Maize MATE Polypeptide MATE EP1

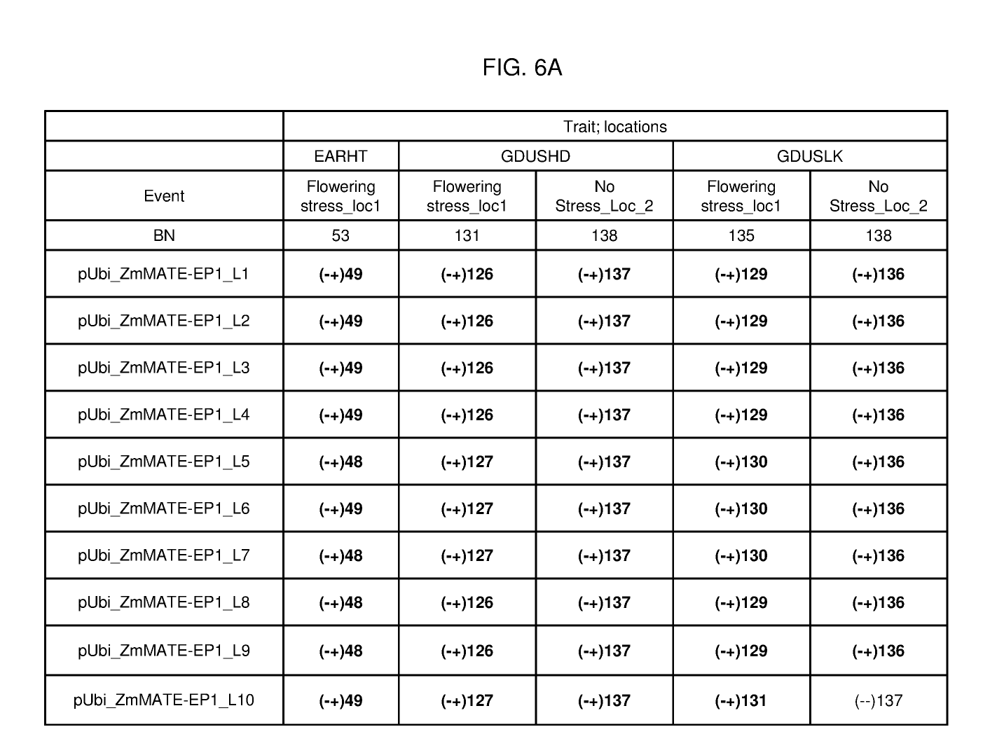

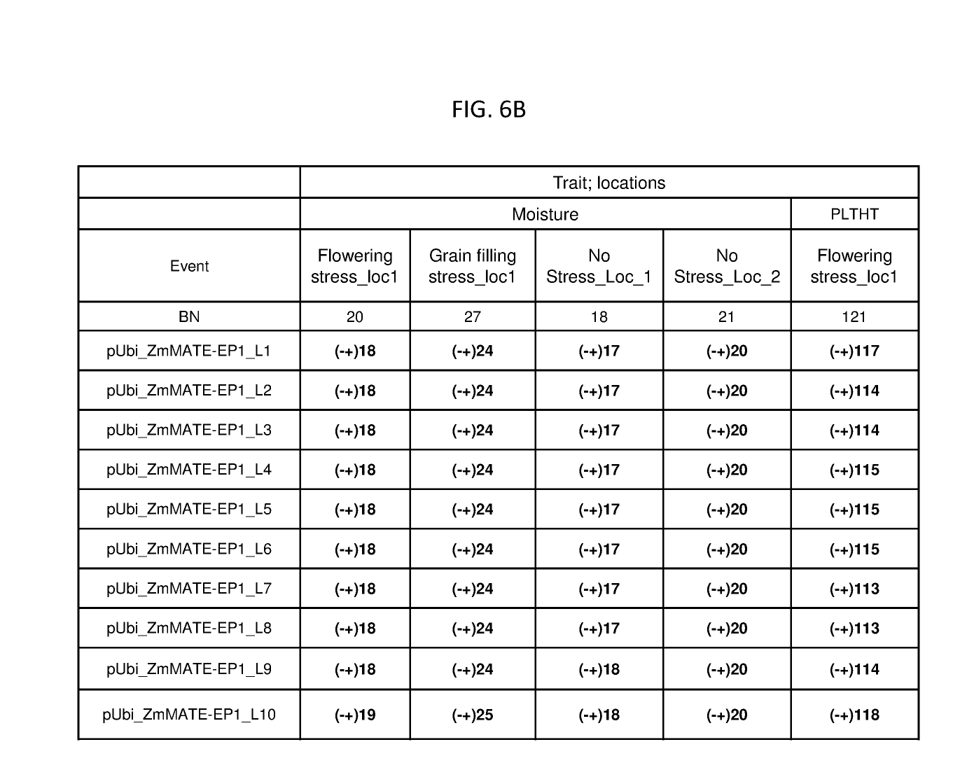

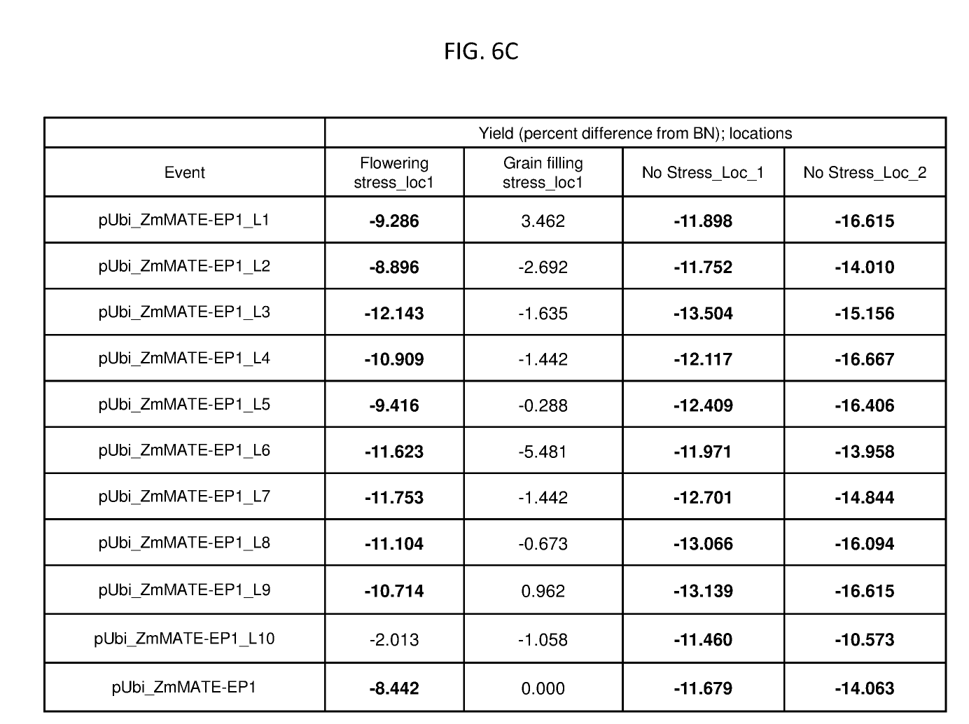

Ten transgenic events for the construct pUbi-ZmMATE_EP1 encoding the maize MATE polypeptide MATE_EP1 (SEQ ID NO:6) were field tested for different traits at several locations, Ear height (EARHT) was tested at a location with drought stress at flowering (FIG. 6A), GDU to shed and GDU to silk (GDUSHD and GDUSLK) were tested at locations with flowering stress and no-stress location, and there was a reduction in ear height compared to bulk null. GDUSHD and GDUSLK were found to be significantly reduced in all events compared to BN. Grain moisture content was tested at 4 locations: two locations without stress, one location with drought stress at flowering stage, and one location with drought stress at grain-filling stage (FIG. 6B), and was found to be reduced in all locations for all events. Plant height was also tested at the location with drought stress at flowering (FIG. 6B), and was found to be reduced compared to the bulk null. Statistical significance is reported at P<0.1 for a two-tailed test.

The significant values (with p-value less than or equal to 0.1 with a 2-tailed test) are shown in bold.

Yield data was also located for 4 locations, two locations without stress, one location with drought stress at flowering stage, and one location with drought stress at grain-filling stage (FIG. 6C). FIG. 6C shows the field data with yield analysis for the 10 events (yield is shown as percent difference from the bulk null). Negative effect of the transgene was observed in three out of four locations, for multiple events.

What is claimed is:

1. A plant comprising in its genome a polynucleotide operably linked to at least one heterologous regulatory element, wherein said polynucleotide encodes a MATE-efflux polypeptide, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising said recombinant DNA construct.

2. The plant of claim 1, wherein said polynucleotide encodes a MATE-efflux polypeptide having an amino acid sequence that has at least 80% sequence identity, when compared to SEQ ID NO:2, 4, 6, 8, 10, 12, 13, 14, 15, 16, 17, 18 or 20.

3. The plant of claim 1, wherein said polynucleotide encodes a MATE-efflux polypeptide comprising the amino acid sequence of SEQ ID NO:2, 4, 6, 8, 10, 12, 13, 14, 15, 16, 17, 18 or 20.

4. The plant of claim 1, wherein the plant overexpresses said polypeptide.

5. The plant of claim 1, wherein the relative maturity of plant is reduced.

6. Seed of the plant of claim 1, wherein said seed comprises in its genome a recombinant DNA construct comprising a polynucleotide operably linked to at least one heterologous regulatory element, wherein said polynucleotide encodes a MATE-efflux polypeptide having an amino acid sequence of at least 80% sequence identity, when compared to SEQ ID NO:2, 4, 6, 8, 10, 12, 13, 14, 15, 16, 17, 18 or 20, and wherein a plant produced from said seed exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising said recombinant DNA construct.

7. The plant of claim 1, wherein the polynucleotide comprises a nucleotide sequence that has at least 80% sequence identity, when compared to SEQ ID NO:1, 3, 5, 7, 9 or 19, and wherein the polynucleotide sequence can be modified by CRISPR-Cas.

8. A plant comprising in its genome an endogenous polynucleotide operably linked to at least one heterologous regulatory element, wherein said endogenous polynucleotide encodes a MATE-efflux polypeptide having an amino acid sequence that has at least 80% sequence identity, when compared to SEQ ID NO:2, 4, 6, 8, 10, 12, 13, 14, 15, 16, 17, 18 or 20, and wherein said plant exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length and reduced grain moisture, when compared to a control plant not comprising the heterologous regulatory element operably linked to the endogenous polynucleotide.

9. The method of claim 8, wherein the at least one heterologous regulatory element is at least one regulatory element endogenous to the plant.

10. (canceled)

11. The plant of claim 10, wherein the monocot plant is a maize plant.

12. (canceled)

13. (canceled)

14. (canceled)

15. The method of claim 13, wherein the plant is a monocot plant.

16. The method of claim 15, wherein the monocot plant is a maize plant.

17. (canceled)

18. (canceled)

19. A method of obtaining a plant that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, the method comprising the steps of:
(a) introducing a mutation into an endogenous MATE-efflux gene of a plant, to create a mutant plant comprising a MATE-efflux mutant gene; and
(b) obtaining the mutant plant of step (a) that exhibits at least one altered agronomic characteristic, wherein the altered agronomic characteristic is selected from the group consisting of: shorter plant stature, reduced days to shed, earlier flowering, reduced days to silk, earlier senescence, shorter life cycle, increased leaf number, reduced stalk diameter, hypersensitivity to day length, and reduced grain moisture, when compared to a control plant not comprising the MATE-efflux mutant gene.

20. The method of claim 19, wherein step (a) is done using at least one method selected from the group consisting of: Targeting Induced Local Lesions IN Genomics (TILLING), transposon tagging, and Cas9 nuclease/guide-RNA mediated genome editing.

21. The method of claim 19, wherein the mutation is in a non-coding region of the MATE-efflux gene.

22. (canceled)

23. (canceled)


SEQUENCE LISTING (21 sequences)

SEQ ID NO: 1 - DNA, 1518 bp, Zea mays
atggccatcccgctgccgtgcaaggcgctgccgcggcatgacgggacgaagggcagcgggcaggcgtacgacggcgacgaccagccgtcggtggctgcggagctgcgcgcgctgtggggcatggcggcgcctgtcactgctctcaactgcgtggtgtacctgcgcgcgatggtgtccgtgctgtgcctggggcggctgggcccgctggacctggcgggcggcgcgctggccatcgggctgaccaacatcacgggccacagcgtgctgttcgggctggcgtcggggctggagccgctgtgcgcgcaggcgttcgggtcccgcaaccacgagctgctgacgctgtccctccagcgcgccgtgctgctgcttctcctggcggcgctgcccatcgcgctgctgtggctgaacgtgggccccatcctggtggcgctggggcaggaccccaccatctcggcgcacgccgcgtcgtacgccgcgttcgcgctcccggaccttgccgcgggcgccgtgctccagccgctgcgcgtgtacctccggtcgcagggcatcacgcgccccatggcggcctgctccgccgccgccgtggcgctgcacgtcccgctcaacgtcggcctcgtcttcggcttgggcctcggcgtgcgcggcgtcgcggcggcgcaggccctcaccaacgccaacatgctgctcttcctgctcgcctacgtgcgctgggcgcgcgcctgcgagcacacctggaagggctgggcgcgccccgccgacgtcgccgccggcctcccggccctcgccggcctcgccgtgcccagctgcgtcggcgtctgcctcgagtggtggtggtacgaggtcgtcaccgtgctcgccggctacctccccaaccccgccgccgcagttggcgccgccggggtgctcatccagaccaccagcctcatgtacaccgtgcccatggcgctcgccgccagcgtctctacccgggtgggcaacgagctgggcgccgggaagccgcggcgcgcccggatggcggcgctggtggcgctgtggtgcgcgctggcggtcggcgtgctgcacgtggcgtggacggtggcgctgagcaggcggtgggtggagctgttcacggcggatcccggcgtggtgcggctggcgtcggcggcgatgcccgtggtggggctgtgcgagctgggcaactgcccgcagacggcgggctgcggggtgctccggggcacggcgcgcccggccgtgggcgcccgcatcaacctcctctccttctacctggtgggcacccccgtggcggtgtacctggcgttcggcgcccgcgccgggttccgcggcctctggtacggcctcctgtcggcgcaggccacgtgcgtggcgctcgtcctcgtcgccgtcgtctggcgcacggactggcgcgtcgaggccatgcgcgccaagaagctggcggggctggagctcaccaacgacgccgaggaggagagcaggcgcctcgtggccgccaccggcgagcaggcggaggatactagctag

SEQ ID NO: 2 - protein, 505 aa, Zea mays
MAIPLPCKALPRHDGTKGSGQAYDGDDQPSVAAELRALWGMAAPVTALNCVVYLRAMVSVLCLGRLGPLDLAGGALAIGLTNITGHSVLFGLASGLEPLCAQAFGSRNHELLTLSLQRAVLLLLLAALPIALLWLNVGPILVALGQDPTISAHAASYAAFALPDLAAGAVLQPLRVYLRSQGITRPMAACSAAAVALHVPLNVGLVFGLGLGVRGVAAAQALTNANMLLFLLAYVRWARACEHTWKGWARPADVAAGLPALAGLAVPSCVGVCLEWWWYEVVTVLAGYLPNPAAAVGAAGVLIQTTSLMYTVPMALAASVSTRVGNELGAGKPRRARMAALVALWCALAVGVLHVAWTVALSRRWVELFTADPGVVRLASAAMPVVGLCELGNCPQTAGCGVLRGTARPAVGARINLLSFYLVGTPVAVYLAFGARAGFRGLWYGLLSAQATCVALVLVAVVWRTDWRVEAMRAKKLAGLELTNDAEEESRRLVAATGEQAEDTS

SEQ ID NO: 3 - DNA, 1722 bp, Zea mays
atggcgacgtgccggtgccccgagccggcgcaatgccatcacaggccgctcctctcgacggccaagggctaccctaagctgcacgaccgcccaaggccggcgcccggcagcgtcctcgccgaggtggccgccatcctctgcctcaccgggccaatggccggcgcggggatcctgttctacatgcgctccctcgtgtccatggtgttcctcggccgcctcggccagctgcccctcgccggcggctccctcgcgctcggcttcgccaacatcaccggctattccgtcctctccggcctcgccggcgggatggacccggtctgcggccaggcgttcggcgcggggcggacggacctcctccgcaccgcactccggcgcactgttctgctgctcctggccgcgtccgtgcccatcgccatgctctgggtggccatgaaccgggtcctcgtctccgcggggcaggaccctgacattgcggccacggcgtacgcctacatcctctgctgcctccccgacctcgtgctgcagtgcttcctccacccgatccgtatctacctgcgcgcgcagtccgtcacgctgccgctcacctacggcgccgccgccgcgctgctcctccacgtgcccatcaacttcctcctcgtcagcgtcctcggcctcggcgtccgtggcgtcgcgctcggcggggtctggaccaacctcaacttcttgctgttcctcgtggcgtacgtctacctccgcggaatgtacggcacccatgacgatgacggcgccaagaagggcgcgtccgtggcggcgccaccagccgaggaggtggaggaggagggcgccaaggagtggtggagcctggtgaggctgtgcgcgcacagctgcatgtccgtctgcttggagtggtggtggtacgagatcatggtgctcctctgtggcgtgctcatcgaccccaaggccgcggtggcttccatgggggtgctcatacagaccacgtcgctgatatacatcttcccgcactcgctcggctgcgccgtgtccacgcgagtcgggcacgagctgggcgcccggaggccggagcgcgcgcgcctcgtcgcgcgcgtcgggctcggcctgggcgccgcgctcggcctcgtggcctgcgcattcgccgtgtccgtgcggggcgtctgggcacggatgttcaccaccgacgaggccatcctgaagctggcggcggcagcgctaccgctgctcggcatggccgaactggggaactgcccgcagacggccggctgcggggtgctgcgcggcagcgcgaggcccgggaaggcggcgaggatcaacgtctcggcgttctacggcgtgggcatgcccgtggcgctggcgctggcgttctgggcccccgggctggacttccgcgggatgtggggcggcatgctggccgcgcagctcgtctgcgccgcgctcatgctgcgcgccgtgctcggcaccgactgggcggagcagacggagcgcgcccgccagctcaccggcgggcgcggcgacggctttgacgtcgtcggcattgtcgacgaggacaaaacaagccatgcagaagcggccaaagcagaggtcgggaacacattgttcatagtggcggattgcgtgattgcgtctagcggcgacggaaccaggacccgattcatggatgatttgcatcctcgattcattagatataggttttcttttttctttattagttttttttcctttttgtaa

SEQ ID NO: 4 - protein, 573 aa, Zea mays
MATCRCPEPAQCHHRPLLSTAKGYPKLHDRPRPAPGSVLAEVAAILCLTGPMAGAGILFYMRSLVSMVFLGRLGQLPLAGGSLALGFANITGYSVLSGLAGGMDPVCGQAFGAGRTDLLRTALRRTVLLLLAASVPIAMLWVAMNRVLVSAGQDPDIAATAYAYILCCLPDLVLQCFLHPIRIYLRAQSVTLPLTYGAAAALLLHVPINFLLVSVLGLGVRGVALGGVWTNLNFLLFLVAYVYLRGMYGTHDDDGAKKGASVAAPPAEEVEEEGAKEWWSLVRLCAHSCMSVCLEWWWYEIMVLLCGVLIDPKAAVASMGVLIQTTSLIYIFPHSLGCAVSTRVGHELGARRPERARLVARVGLGLGAALGLVACAFAVSVRGVWARMFTTDEAILKLAAAALPLLGMAELGNCPQTAGCGVLRGSARPGKAARINVSAFYGVGMPVALALAFWAPGLDFRGMWGGMLAAQLVCAALMLRAVLGTDWAEQTERARQLTGGRGDGFDVVGIVDEDKTSHAEAAKAEVGNTLFIVADCVIASSGDGTRTRFMDDLHPRFIRYRFSFFFISFFSFL

SEQ ID NO: 5 - DNA, 1428 bp, Zea mays
atggatgaaccccttcttggcaatggtcttaagaccagcgggaaaagagagagcctggtagtggccgaggtcagaaaacagatgtaccttgctgggccactcatcgctgcatggatcctacagaacattgtccagatgatatctatcatgtttgtcggtcaccttggtgagcttgccctctccagtgcctccatcgccacctcctttgcaggcgttactggcttcagcttattgtctggcatggcgagcagcttggacacactgtgtgggcaatcgttcggggcaaagcagtactatcttcttggcatctacaagcagagggcaatccttgtgctcactctagtcagccttgtggttgcgattatctggtcatacactggacagatccttctactctttggtcaggatccagagattgcagctggggcagggagctatatccggtggatgattcctgcgctatttgtgtatggtccactacagtgccatgtccggtttctgcaaacgcagaacatagtcctcccagtgatgctgagctcaggtgccacagcactgaaccatctgctggtgtgctggctgctggtgtacaagattggtatgggcaacaagggtgctgccttggccaatgccatctcatactttaccaatgtatcaatcctggcaatttacgtcaggcttgcaccagcctgtagaaacacctggagagggttctcaaaggaggcttttcatgacataaccagcttcttgaggcttggtattccatctgcgctgatggtttgcttggagtggtggtcatttgagctcctggtacttctttctggacttctccccaatccaaagcttgagacatcggttttgtccatttccttaaacacaggctctttagcgtttatgatcccttttgggcttagcgcagccataagcacccgtgtttcaaatgaacttggtgctgggcgaccccatgctgcccatctggctacccgtgtggtcatggtgctggccatcgtggttggcgtattaatcggactagctatgattttggtgcgcaatatctgggggtatgcatacagtaatgagaaggaggtggtcaaatacatctccaaaatgatgccgatcctagccgtgtcattcttgttcgattgcgtgcagtgtgttctttcaggtgttgctaggggctgtggatggcaaaagattggggcttgtgttaatcttggtgcatactaccttataggaattccagctgccttctgtttcgcctttatgtaccatctaggtggaatgggactttggttgggaataatctgcgcactcgtcatacagatgctattgcttctcacaattaccttgtgcagcaactgggagaaagaagctttgaaggcaaaggacagagtatttagtacatccgtaccagctgacatgatgacatga

SEQ ID NO: 6 - protein, 475 aa, Zea mays
MDEPLLGNGLKTSGKRESLVVAEVRKQMYLAGPLIAAWILQNIVQMISIMFVGHLGELALSSASIATSFAGVTGFSLLSGMASSLDTLCGQSFGAKQYYLLGIYKQRAILVLTLVSLVVAIIWSYTGQILLLFGQDPEIAAGAGSYIRWMIPALFVYGPLQCHVRFLQTQNIVLPVMLSSGATALNHLLVCWLLVYKIGMGNKGAALANAISYFTNVSILAIYVRLAPACRNTWRGFSKEAFHDITSFLRLGIPSALMVCLEWWSFELLVLLSGLLPNPKLETSVLSISLNTGSLAFMIPFGLSAAISTRVSNELGAGRPHAAHLATRVVMVLAIVVGVLIGLAMILVRNIWGYAYSNEKEVVKYISKMMPILAVSFLFDCVQCVLSGVARGCGWQKIGACVNLGAYYLIGIPAAFCFAFMYHLGGMGLWLGIICALVIQMLLLLTITLCSNWEKEALKAKDRVFSTSVPADMMT

SEQ ID NO: 7 - DNA, 1809 bp, Zea mays
atgaaccatttccctgttgggcccatggaaaatccaaacgatagaatgtcatgcgctgcacctgcaagcatctatataaacagtgccagtacgagtgccagctctcgtccctgccaccgcaacagcacacagacgtggcccctctgcggcaaacaaaccagaggccgcggcaggcacggggacacttcacgcaggccgcgcagcagcgccatggcgacgtgccagtgcccggagccggcgcaatgccaccacaggccgctcctctcgtcgacggccgacacctaccctgagctgcacgaccgcccgaggcctgcgcccggcagcgtcctcgccgaggtggccgccatcctctgcctcacggggccaatggtcggcgcgggcatcctgttctacatgcgctcgctggtgtccatggtgttcctcggccgcctcggccagctgcccctcgccgggggctccctcgcgctcggcttcgcaaacatcaccggctactccgtcctctcagggctcgccggcgggatggaccccgtctgcggccaggcgttcggcgcggggcggacggacctcctccgcgccgcgctccggcgcaccgttctgctgctcctggccgcgtccgtgcccatcggcctgctctgggcggccatgcaccgggtcctcgtctccacggggcaggaccccgacatcgcggccaccgcgtacgcctacatcctctgctgcctccccgacctcgtgctgcagtgcttcctccacccgatccgcatctacctccgcgcgcagtccgtcacgctgccgctcacctacggcgccgccgccgcgctgctcctccacgtgcccatcaacttcctcctcgtgagcggcctcggcctcggcgtccgtggcgtcgcgctcggaggggtctggaccaacctcaacttcttgcttttcctcgtggcgtacgtctacttccgcggaatgtacggcgcccatgacgacggcggcgccaagaagggcgcgtccgtggcgccgccgacaccagccgagggggagtggtggagccttgtgaggctgtgcgtgcacagctgcatgtcggtctgcctggagtggtggtggtacgagatcatggtgctcctctgtggcgtgctcatcgaccccaaggcggcggtggcggccatgggcgtgctcatacagaccacctcgctgatatacatcttcccgcattcgctcggctgcgccgtttccacgcgtgtcgggcacgagctgggcgcccggaggccggagcgcgcgcgcctcgtcgcgcgcgtcgggctcggcctgggcgccgcgctgggcctggtggcctgcgcattcgccgtgtccgtgaggggcatctgggcgcggatgttcaccgcggacgaggccatcctgaggctggcggcggcggcgctgccgctgctcggcacggccgagctggggaactgcccgcagacggccggctgcggcgtgctgcgcggcagcgcgcggcccgagaaggcggcgaggatcaacgtgtccgcgttctacggcgtgggcatgcccgtggcgctggcgctggcgttctggcccgcccgtctggacttccgcgggatgtggggcggcatgctggccgcgcagctcgtctgcgccgcgctgatgctgcgcgccgtgctcggcaccgactgggcggagcagacggagcgcgcccgccagctcaccggcgggcgcggcggcggctttgacgtcgtcggcattgtcgacgaggacaaaacaagccatgcagaagcggccaaagcagaggtcgggaacacactgttcgtggtggcggattgcgtctag

SEQ ID NO: 8 - protein, 602 aa, Zea mays
MNHFPVGPMENPNDRMSCAAPASIYINSASTSASSRPCHRNSTQTWPLCGKQTRGRGRHGDTSRRPRSSAMATCQCPEPAQCHHRPLLSSTADTYPELHDRPRPAPGSVLAEVAAILCLTGPMVGAGILFYMRSLVSMVFLGRLGQLPLAGGSLALGFANITGYSVLSGLAGGMDPVCGQAFGAGRTDLLRAALRRTVLLLLAASVPIGLLWAAMHRVLVSTGQDPDIAATAYAYILCCLPDLVLQCFLHPIRIYLRAQSVTLPLTYGAAAALLLHVPINFLLVSGLGLGVRGVALGGVWTNLNFLLFLVAYVYFRGMYGAHDDGGAKKGASVAPPTPAEGEWWSLVRLCVHSCMSVCLEWWWYEIMVLLCGVLIDPKAAVAAMGVLIQTTSLIYIFPHSLGCAVSTRVGHELGARRPERARLVARVGLGLGAALGLVACAFAVSVRGIWARMFTADEAILRLAAAALPLLGTAELGNCPQTAGCGVLRGSARPEKAARINVSAFYGVGMPVALALAFWPARLDFRGMWGGMLAAQLVCAALMLRAVLGTDWAEQTERARQLTGGRGGGFDVVGIVDEDKTSHAEAAKAEVGNTLFVVADCV

SEQ ID NO: 9 - DNA, 1599 bp, Zea mays
atggcgacgtgccagtgcccggagccggcgcaatgccaccacaggccgctcctctcgtcgacggccgacacctaccctgagctgcacgaccgcccgaggcctgcgcccggcagcgtcctcgccgaggtggccgccatcctctgcctcacggggccaatggtcggcgcgggcatcctgttctacatgcgctcgctggtgtccatggtgttcctcggccgcctcggccagctgcccctcgccgggggctccctcgcgctcggcttcgcaaacatcaccggctactccgtcctctcagggctcgccggcgggatggaccccgtctgcggccaggcgttcggcgcggggcggacggacctcctccgcgccgcgctccggcgcaccgttctgctgctcctggccgcgtccgtgcccatcggcctgctctgggcggccatgcaccgggtcctcgtctccacggggcaggaccccgacatcgcggccaccgcgtacgcctacatcctctgctgcctccccgacctcgtgctgcagtgcttcctccacccgatccgcatctacctccgcgcgcagtccgtcacgctgccgctcacctacggcgccgccgccgcgctgctcctccacgtgcccatcaacttcctcctcgtgagcggcctcggcctcggcgtccgtggcgtcgcgctcggaggggtctggaccaacctcaacttcttgcttttcctcgtggcgtacgtctacttccgcggaatgtacggcgcccatgacgacggcggcgccaagaagggcgcgtccgtggcgccgccgacaccagccgagggggagtggtggagccttgtgaggctgtgcgtgcacagctgcatgtcggtctgcctggagtggtggtggtacgagatcatggtgctcctctgtggcgtgctcatcgaccccaaggcggcggtggcggccatgggcgtgctcatacagaccacctcgctgatatacatcttcccgcattcgctcggctgcgccgtttccacgcgtgtcgggcacgagctgggcgcccggaggccggagcgcgcgcgcctcgtcgcgcgcgtcgggctcggcctgggcgccgcgctgggcctggtggcctgcgcattcgccgtgtccgtgaggggcatctgggcgcggatgttcaccgcggacgaggccatcctgaggctggcggcggcggcgctgccgctgctcggcacggccgagctggggaactgcccgcagacggccggctgcggcgtgctgcgcggcagcgcgcggcccgagaaggcggcgaggatcaacgtgtccgcgttctacggcgtgggcatgcccgtggcgctggcgctggcgttctggcccgcccgtctggacttccgcgggatgtggggcggcatgctggccgcgcagctcgtctgcgccgcgctgatgctgcgcgccgtgctcggcaccgactgggcggagcagacggagcgcgcccgccagctcaccggcgggcgcggcggcggctttgacgtcgtcggcattgtcgacgaggacaaaacaagccatgcagaagcggccaaagcagaggtcgggaacacactgttcgtggtggcggattgcgtctag

SEQ ID NO: 10 - protein, 532 aa, Zea mays
MATCQCPEPAQCHHRPLLSSTADTYPELHDRPRPAPGSVLAEVAAILCLTGPMVGAGILFYMRSLVSMVFLGRLGQLPLAGGSLALGFANITGYSVLSGLAGGMDPVCGQAFGAGRTDLLRAALRRTVLLLLAASVPIGLLWAAMHRVLVSTGQDPDIAATAYAYILCCLPDLVLQCFLHPIRIYLRAQSVTLPLTYGAAAALLLHVPINFLLVSGLGLGVRGVALGGVWTNLNFLLFLVAYVYFRGMYGAHDDGGAKKGASVAPPTPAEGEWWSLVRLCVHSCMSVCLEWWWYEIMVLLCGVLIDPKAAVAAMGVLIQTTSLIYIFPHSLGCAVSTRVGHELGARRPERARLVARVGLGLGAALGLVACAFAVSVRGIWARMFTADEAILRLAAAALPLLGTAELGNCPQTAGCGVLRGSARPEKAARINVSAFYGVGMPVALALAFWPARLDFRGMWGGMLAAQLVCAALMLRAVLGTDWAEQTERARQLTGGRGGGFDVVGIVDEDKTSHAEAAKAEVGNTLFVVADCV

SEQ ID NO: 11 - protein, 514 aa, Hordeum vulgare
MAVPLQAKKQQENGSKGCQGSDMDGGDDHPSAAEELRELWRMAAPITALNCVVYLRAMVSVLCLGRLGPLDLAGGALAIGLTNITGHSVLFGLASGLEPLCAQAYGSRNYDMLTLSLHRAVLLLAIAAVPIALLWLNVGPILVALGQDPAISASAAAYAAWALPDLAALAVLQPLRVYLRSQGITKPMAACSAAAVALHVPLNVLLVFKMGFGVRGVAAAQALTNTNMVLFLLAYVRWAGACDDTWRGFARPAAVASGLGGLVRLAVPSCIGVCLEWWWYEVVTVLAGYLPNPTAAVGAAGVLIQTTSLMYTVPMALAACVSTRVGNELGAGKPRRARMAATVALWCAAGVGLAHVAWTATFSAQWVSLFTREPSVLLLASAAMPVLGLCELGNCPQTTGCGVLRGTARPAVGARINLLSFYLVGTPVAVLLAFGPGPRAGFGGLWYGLLSAQAACVALVLVAVVWRTDWRVEAMRARRLTGTGAETTATATEGDQEEMRRLVAGNGREADVDV

SEQ ID NO: 12 - protein, 524 aa, Brachypodium distachyon
MATCHCSEPAQCLHRPLLTPAPGYPRLHDRKKTAAAGGGGGVVGEVASILRLTGPMVGAGILLYLRSLVSMVFLGRLGQLPLAGGSLALGFANITGYSVLSGLAGGMDPVCGQAFGAGRTDLLRAALRRTVALLLAASVPISALWLAMRRVLVATGQDADIASVAYAYILCSLPDLVVQSFLHPLRIYLRAQSVTLPLTYAAAAAVLLHVPINFVLVDVLGLGIRGVALGAVCTNLNFLLFLVAYVCVFGMYGGEDSDKKAATPVEEEEGAREWWSLVRLSVHSCMSVCLEWWWYEIMVLLCGVLADPKAAVAAMGVLIQTTSLIYIFPHSLGCAVSTRVGNELGAGRPERARLVARVGLCLGAGLGLVACAFAVSVRGVWARTFTTDDAILKLTAAALPLLGLAELGNCPQTTGCGVLRGSARPEKAAKINVSAFYGVGMPVALALAFRPARLDFRGMWGGMLAAQLVCAALMLRAVLGTDWDEQTERARELTGGGIVDDKQHAEVAKGGADNALFVVVADCV

SEQ ID NO: 13 - protein, 516 aa, Oryza sativa
MAIPLQGKAQQQQGEGGKGGGAADDDGDDQPSVASELRELWGMAAPITALNCVVYLRAMVSVLCLGRLGPLDLAGGALAIGLTNITGHSVLFGLASGLEPLCAQAFGSKNYDLLTLSLQRAVLLLTLAALPIALLWLHVGPILVALGQDPTISASAAAYAAYALPDLAASAVLQPLRVYLRSQGITKPMAACSAIAVALHVPLNVLLVFGLGFGVRGVAAAQALTNTNMVLFLLAYIRWSRACDATWKGWARPAAVASGLAGLVRLAVPSCVGVCLEWWWYEVVTVLAGYLPNPAAAVGAAGVLIQTTSLMYTVPMALAACVSTRVGNELGGGKPRRARMAAMVALGCAVVIGVVHVAWTAAFSREWVELFTREAAVVRLAAAAMPILGLCELGNCPQTTGCGVLRGTARPAVGARINLLSFYLVGTPVAVTLAFGARVGFGGLWYGLLSAQAACVALVLLAVVWRTDWHLEALRAKKLTGLEMITAAAGDDDECKRLIAPLPPPDGHDVAIVDVV

SEQ ID NO: 14 - protein, 504 aa, Setaria italica
MAIPLQGKDGKGGGQVGSDDHPSATAELRALWGMAAPITALNCVVYLRAMVSVLCLGRLGPLDLAGGALAIGLTNITGHSVLFGLASGLEPLCAQAFGSRNHDLLTLSLQRAMLLLFLAALPIALLWLNVGPILVALGQDPAISAPAAAYARFALPDLAASVVLQPLRVYLRSQGITRPMAACSAIAVALHVPLNVLLVFGLGFGVRGVAAAQALTNTNMLLFLLAYIRWARACDDTWRGWARITAVASGLPELARLAVPSCVGVCLEWWWYEVVTVLAGYLPNPAAAVGGAGVLIQTTSLMYTVPMALAACVSTRVGNELGAGKPRRARMAAMVALACALAIGAVHVAWTAALSRQWVELFTTEPAVVRLAAAAMPIVGLCELGNCPQTTGCGVLRGTARPAVGARINLLSFYLVGTPVAVYLAFGAGAGFSGLWYGLLSAQATCVALILAAVVWRTDWQVEAMRAKKLAGLELAPTTTAATTTNDAESKRLVAANGEPAEDV

SEQ ID NO: 15 - protein, 529 aa, Sorghum bicolor
MAIPLPGKALPRHDVSKGGGQADADDAAAWPGGDDDQPSVVAELRALWGMAAPITALNCVVYLRAMVSVLCLGRLGPLDLAGGALAIGLTNITGHSVLFGLASGLEPLCAQAFGSRNHELLTLSVQRAVLLLFLAAVPIALLWLNVGPILVALGQDPTISAHAASYAAFALPDLAAGAVLQPLRVYLRSQGITRPMAACSAIAVVLHVPLNVALVFGMGLGVRGVAAAQALTNTNMLLFLLAYIRWARACEGTWKGWARPAAVASGLPALASLAVPSCVGVCLEWWWYEVVTVLAGYLPNPAAAVGAAGVLIQTTSLMYTVPMALAACVSTRVGNELGAGKPRRARMAALVALWCALAIGVVHVAWTVALSRRWVELFTTEAGVVRLASAAMPVVGLCELGNCPQTTGCGVLRGTARPAVGARINLLSFYLVGTPVAVYLAFGAPGVGFRGLWYGLLSAQASCVALVLAAVVWRTDWRVEAMRAKKLAGLELAHPVPTTTAADDADDAAEERKRLVVAGTGEPAAEGGV

SEQ ID NO: 16 - protein, 527 aa, Hordeum vulgare
MATCHCSEPAQCLHQALLVPDRAYPRLRDRKTTTVPAGDGVLAEVASILCLTAPIVGAGILLYLRSLVSMVFLGRLGQLPLAGGSLALGFANITGYSVLSGLAGGMDPVCGQAFGAGRTDLLRAALRRTVVLLLIASVPISVLWVAMHRVLVATGQDPDIASTAYAYILCSLPDLAVQSFLHPLRIYLRAQSVTLPLTYAAAAAVLLHVPINFVLVDVLGLGIRGVALGAVCTNLNFLLFLVAYVCFFGMYGHDDGETKASAAAAEDESAKEWWSLVRLSVHSCMSVCLEWWWYEIMVLLCGVLADPKAAVAAMGVLIQTTSLIYIFPHSLSCAVSTRVGHELGARRPERARLVARVGLGLGAVLGLVACAFAVSVRGVWARMFTADDAILRLTAAALPLLGLAELGNCPQTTGCGVLRGSARPEKAARINVSAFYGVGMPVALLLAFRPGRLDFRGMWGGMLAAQLVCAALMLRAVLATDWEEQTNRARELTGGNAGDVVDGDKRKHAEAAKADADNSSLVLADCV

SEQ ID NO: 17 - protein, 541 aa, Oryza sativa
MASRHSDEATQCHQQLLVMPAATASYPKLHDRPRLAGAAAGVLGEVASILCLAGPMVGAGILLYLRSLVSMVFLGRLGQLPLAGGSLALGFANITGYSVLSGLAGGMDPVCGQAFGAGRTDLLRAALRRTVVLLLAASVPISALWVAMHRVLVATGQDPDIAATAYAYILCSLPDLAVQCFLHPIRIYLRAQSVTLPLTYAAAAALLLHVPINVVLVDRLGLGIRGVALGAVCTNLNCLLFLAAYVCLSGMYGGRAKACASAAAPAAGEEDDDGGVREWWSLVRLSVHSCMSVCLEWWWYEIMVLLCGVLADPKAAVAAMGVLIQTTSLIYIFPHSLGCAVSTRVGHELGAGRPERARLVARVGVGLGAALGIVAFGFAVSVRAAWARMFTAEDAILRLAAAALPLLGAAELGNCPQTAGCGVLRGSARPERAARINVAAFYGVGMPVALALAFWPAGLDFRGMWGGMLAAQLVCAWLMLRAVLGTDWAEQAERARELTGGGDGYAAVAAVIVDDDKAKQHAEMDKPQQVDNTLLMAIDCV

SEQ ID NO: 18 - protein, 547 aa, Sorghum bicolor
MATCQCPELAQCHHRALLPTAKSYPKLHDRPRPAPGTGTGTGSVLAEVAAILCLTGPMVGAGILFYMRSLVSMVFLGRLGQLPLAGGSLALGFANITGYSVLSGLAGGMDPVCGQAFGAGRTDLLRTALRRTVLLLLAACVPIAMLWVAMHRVLVSTGQDPDIAATAYAYILWCLPDLVLQCFLHPIRIYLRAQSVTLPLTYGAAAALLLHVPINFLLVSVLGLGIRGVALGGVWTNLNFLLFLVAYVYLRGMYGAHDDDGSAKKGGASVAAPPPAEEEVGANSKEWWSLVRLCVHSCMSVCLEWWWYEIMVLLCGVLIDPKAAVAAMGVLIQTTSLIYIFPHSLGCAVSTRVGHELGARRPERARLVARVGLGLGAALGLVACAFAVSVRGVWARMFTADEAILKLAAAALPLLGMAELGNCPQTAGCGVLRGSARPEKAARINVSAFYGVGMPVALALAFWPAGLDFRGMWGGMLAAQLVCAALMLRAVLGTDWAEQTERARQLTGGGRGDGFDVVGIVDEDKTSHAEAAKAEVGNALFMVADCV

SEQ ID NO: 19 - DNA, 1506 bp, Arabidopsis thaliana
atgaactccgaatccctggagaacctgcaccgccctctgatcgagtcgtccaagagcttcgtggattaccgcttggagaccgtgctgactgaccgcgagctcccatacttccgcaggatctacctcgccatgatgatcgagatgaagttcctgttccacctcgctgcgccggcaatcttcgtctacgtcatcaacaacgggatgagcatcctgacccggatcttcgcaggtcatgtgggcagcttcgagcttgcagctgcatccctgggcaactccggattcaacatgttcacctacggcctcctccttggtatgggctcggcagtggaaaccctgtgtggacaagcacacggagctcacaggtacgagatgctgggcgtgtacctgcagcggtccaccgtggtgcttatcctcacgtgtctcccgatgtccttcctcttcctgttctccaacccgatactgaccgcacttggcgagcccgagcaggtcgctactctcgcttcggtgttcgtgtacgggatgatcccggtcatcttcgcgtacgccgtcaacttcccgatccagaagtttctccagtcccagagcatcgtcactcccagcgcgtacatctcggcggccaccctggtcattcacctcatcctcagctggatcgccgtttacagactgggctacggcctcctcgcgctctcactcatacactccttcagctggtggatcatcgtggtcgcccagattgtttacatcaagatgtcaccacggtgtcggagaacctgggaagggtttagctggaaggctttcgagggcctttgggacttcttccggctctccgcagcctctgcagtcatgctgtgtctcgagagctggtactcccagattcttgtgcttctggctgggctcctgaagaatcccgagctcgcgctcgacagcttggcgatctgcatgtcgatctcagccattagcttcatggtgtcggtcggcttcaacgcggcagcatcggtgagggtgtccaacgagctcggagccggaaatccacgggcagcggcctttagcacagtggtgacgactggcgtgtcgtttctcctcagcgtctttgaggctatcgtcgttctctcctggcgccacgtgatcagctacgcattcaccgattcgccggccgtggctgaggctgtcgcagacttgtcacccttcctggcgatcacgatcgtcctgaatggcattcagcctgtcctgtcaggcgtcgcggtgggttgcgggtggcaagccttcgtcgcatacgtcaacattggctgctactacgtggtcggcatcccggtgggcttcgtgctgggattcacctacgatatgggcgccaagggaatctggacgggcatgatcggcgggacccttatgcagacgatcatcctcgtcatcgtcaccctccgcacagactgggacaaggaggtggagaaggcctcgtctcggctcgaccagtgggaagagagcagggagcctctgctcaagcagtag

SEQ ID NO: 20 - protein, 501 aa, Arabidopsis thaliana
MNSESLENLHRPLIESSKSFVDYRLETVLTDRELPYFRRIYLAMMIEMKFLFHLAAPAIFVYVINNGMSILTRIFAGHVGSFELAAASLGNSGFNMFTYGLLLGMGSAVETLCGQAHGAHRYEMLGVYLQRSTVVLILTCLPMSFLFLFSNPILTALGEPEQVATLASVFVYGMIPVIFAYAVNFPIQKFLQSQSIVTPSAYISAATLVIHLILSWIAVYRLGYGLLALSLIHSFSWWIIVVAQIVYIKMSPRCRRTWEGFSWKAFEGLWDFFRLSAASAVMLCLESWYSQILVLLAGLLKNPELALDSLAICMSISAISFMVSVGFNAAASVRVSNELGAGNPRAAAFSTVVTTGVSFLLSVFEAIVVLSWRHVISYAFTDSPAVAEAVADLSPFLAITIVLNGIQPVLSGVAVGCGWQAFVAYVNIGCYYVVGIPVGFVLGFTYDMGAKGIWTGMIGGTLMQTIILVIVTLRTDWDKEVEKASSRLDQWEESREPLLKQ

SEQ ID NO: 21 - protein, 661 aa, Artificial Sequence
  Consensus sequence
  misc_feature (2)..(77): Xaa can be any naturally occurring amino acid
  misc_feature (79)..(79): Xaa can be any naturally occurring amino acid
  misc_feature (86)..(86): Xaa can be any naturally occurring amino acid
  misc_feature (88)..(88): Xaa can be any naturally occurring amino acid
  misc_feature (90)..(95): Xaa can be any naturally occurring amino acid
  misc_feature (98)..(98): Xaa can be any naturally occurring amino acid
  misc_feature (103)..(113): Xaa can be any naturally occurring amino acid
  misc_feature (127)..(128): Xaa can be any naturally occurring amino acid
  misc_feature (137)..(137): Xaa can be any naturally occurring amino acid
  misc_feature (199)..(199): Xaa can be any naturally occurring amino acid
  misc_feature (223)..(223): Xaa can be any naturally occurring amino acid
  misc_feature (229)..(229): Xaa can be any naturally occurring amino acid
  misc_feature (246)..(246): Xaa can be any naturally occurring amino acid
  misc_feature (259)..(259): Xaa can be any naturally occurring amino acid
  misc_feature (275)..(275): Xaa can be any naturally occurring amino acid
  misc_feature (280)..(280): Xaa can be any naturally occurring amino acid
  misc_feature (292)..(292): Xaa can be any naturally occurring amino acid
  misc_feature (305)..(305): Xaa can be any naturally occurring amino acid
  misc_feature (307)..(307): Xaa can be any naturally occurring amino acid
  misc_feature (321)..(321): Xaa can be any naturally occurring amino acid
  misc_feature (329)..(329): Xaa can be any naturally occurring amino acid
  misc_feature (331)..(334): Xaa can be any naturally occurring amino acid
  misc_feature (336)..(336): Xaa can be any naturally occurring amino acid
  misc_feature (347)..(347): Xaa can be any naturally occurring amino acid
  misc_feature (350)..(350): Xaa can be any naturally occurring amino acid
  misc_feature (352)..(358): Xaa can be any naturally occurring amino acid
  misc_feature (366)..(366): Xaa can be any naturally occurring amino acid
  misc_feature (391)..(391): Xaa can be any naturally occurring amino acid
  misc_feature (427)..(427): Xaa can be any naturally occurring amino acid
  misc_feature (511)..(511): Xaa can be any naturally occurring amino acid
  misc_feature (539)..(539): Xaa can be any naturally occurring amino acid
  misc_feature (566)..(566): Xaa can be any naturally occurring amino acid
  misc_feature (584)..(585): Xaa can be any naturally occurring amino acid
  misc_feature (588)..(595): Xaa can be any naturally occurring amino acid
  misc_feature (604)..(604): Xaa can be any naturally occurring amino acid
  misc_feature (613)..(613): Xaa can be any naturally occurring amino acid
  misc_feature (620)..(620): Xaa can be any naturally occurring amino acid
  misc_feature (626)..(661): Xaa can be any naturally occurring amino acid
MXXXXXXXXXXXXXXXXXXXXXXXXXXXXXXXXXXXXXXXXXXXXXXXXXXXXXXXXXXXXXXXXXXXXXXXXXXXXEXAQCHHRXLXLXXXXXXYPXLHDRXXXXXXXXXXXPSVLAEVAAILCLXXPMVGAGILXYLRSLVSMVFLGRLGQLPLAGGSLALGFANITGYSVLSGLAGGMDPVCGQAFGAGRTDLLRXALRRTVLLLLAASVPIALLWLAMXRVLVAXGQDPDIAATAYAYILCXLPDLAAQCFLHPXRIYLRAQSVTLPLTYXAAAAXLLHVPINFLLVXGLGLGVRGVALGXVXTNLNFLLFLVAYVXWRGMYGAXDXXXXTXKGGASVAAPAXGEXEXXXXXXXWWSLVRLXVHSCMSVCLEWWWYEIMVLLCGVLXDPKAAVAAMGVLIQTTSLIYIFPHSLGCAVSTRVGXELGAGRPERARLVARVGLGLGAALGLVACAFAVSVRGVWARMFTADEAILRLAAAALPLLGLAELGNCPQTAGCGVLRGSARPXKAARINVSAFYGVGMPVALALAFWPARXLDFRGMWGGMLAAQLVCAALMLRAVLXGTDWAEQTERARQLTGGXXGDXXXXXXXXIVDEDKTSXHAEAAKAEXGNELFRXVADCVXXXXXXXXXXXXXXXXXXXXXXXXXXXXXXXXXXXX